(19) United States
(12) Patent Application Publication
(10) Pub. No.: US 2024/0122602 A1
(43) Pub. Date: Apr. 18, 2024

(54) VASCULAR OCCLUDER WITH INWARDLY INSERTABLE SEGMENT

(71) Applicant: VenaCore Inc., Wilmington, DE (US)

(72) Inventors: Michael Gabriel TAL, Tel Aviv (IL); Nir HOLTZMAN, Rishon Lezion (IL); Amit GREENER, Tel-Aviv (IL); Navot RABBAN, Ramat-Gan (IL)

(21) Appl. No.: 18/277,506

(22) PCT Filed: Feb. 16, 2022
(86) PCT No.: PCT/US22/16632

§ 371 Date: Aug. 16, 2023

Related U.S. Application Data
(63) Continuation-in-part of application No. PCT/US21/48194, filed on Aug. 30, 2021.
(60) Provisional application No. 63/150,162, filed on Feb. 17, 2021.

(51) Int. Cl. A61B 17/12

(52) U.S. Cl. CPC A61B 17/12172; A61B 17/12036; A61B 2017/00681

(57) ABSTRACT

Disclosed are vascular occluder, kit and method of use thereof. The vascular occluder includes an occluder body extending longitudinally between an occluder body distal end and an occluder body proximal end. The occluder body is elastically compressible longitudinally from a preliminary deployed configuration to a compressed deployed configuration. Vascular occluder includes a tension member configured to tether the occluder body proximal end to the occluder body distal end and to tighten therebetween when the occluder body is in the compressed deployed configuration. The occluder body includes a proximal tubular body segment and a distal tubular body segment, and when in the compressed deployed configuration, the proximal tubular body segment is located inside of, and/or provides structural support to, the distal tubular body segment.

### CROSS-REFERENCE TO RELATED APPLICATIONS

This application claims the benefit of U.S. Provisional Application No. 63/150,162 filed Feb. 17, 2021, and International Application No. PCT/US21/48194 filed Aug. 30, 2021, the entire contents of each of which are incorporated herein by reference.

### FIELD OF THE INVENTION

The present disclosure relates to devices and methods for treating a target blood vessel, and more particularly, but not exclusively, to vascular occluders and methods for occluding target blood vessels such as arteries.

### BACKGROUND OF THE INVENTION

Some medical procedures, such as embolization, involve occluding a blood vessel for reducing pressure on aneurysms, restricting a hemorrhage, or diminishing blood supply to tumors or growths in the body. Vascular plugs are type of mechanical embolization devices commonly used for occluding a targeted portion of vein or artery with a relatively low-profile delivery and can be released in a controlled fashion. Most common are the Amplatzer Vascular Plugs (by St. Jude Medical, Inc.; Minnesota, USA) that have been used for the embolization of medium to large vascular vessels (e.g., greater than 5 mm or greater than 10 mm, respectively) by extending along a relatively long portion of the target vessel, usually greater than 5 mm in length.

In recent years there is a growing trend of micro sized vascular plugs designed for microcatheter delivery, for example the MVP Micro Vascular Plug series (by Medtronic, Plc.; Dublin, Ireland), that include smaller sized vascular plugs deliverable via small-diameter microcatheters but capable of treating vessels smaller than 5 mm in diameter, and vascular plugs of greater size which require relatively larger diameter catheters (4 Fr OD or more) for treating vessels greater than 5 mm in diameter.

There is still an unmet need for mechanical embolization devices (e.g., vascular plugs) capable of meeting one or more of the following main challenges: (a) blocking high flow arteries by applying greater pressure to vessel wall to prevent downstream migration, (b) delivery via small diameter microcatheter for treating a greater range of vessels sizes, such as between 1.5 mm and 10 mm, and (c) increased flexibility for delivering lager-sized vascular plugs in smaller-sized microcatheters.

### SUMMARY OF THE INVENTION

The present disclosure relates to devices and methods for treating a target blood vessel, and more particularly, but not exclusively, to vascular occluders and methods for occluding target blood vessels such as arteries.

In certain embodiments, there is provided vascular occluder comprising an occluder body extending longitudinally between an occluder body distal end and an occluder body proximal end, the occluder body includes a proximal tubular body segment and a distal tubular body segment, and is elastically compressible longitudinally from a preliminary deployed configuration to a compressed deployed configuration. In some embodiments, the vascular occluder includes a tension member configured to tether the occluder body proximal end to the occluder body distal end and to tighten therebetween when the occluder body is in the compressed deployed configuration. In some embodiments, when the occluder body is in the compressed deployed configuration, the proximal tubular body segment is located inside of, and/or provides structural support to, the distal tubular body segment.

In some embodiments, the vascular occluder comprises a proximal occluding section extending between the occluder body proximal end and the proximal tubular body segment, and/or a distal occluding section extending between the occluder body distal end and the distal tubular body segment, configured to cover and/or at least partially occlude most or all cross-sectional area of a passage, when the occluder body is in the compressed deployed configuration.

In some embodiments, the proximal occluding section and/or the distal occluding section includes a meshed, braided and/or porous pattern and/or material across most or all area thereof, configured to reduce flow velocity, and/or to prevent localized increase of flow velocity, of blood passing therethrough, when the occluder body is in the compressed deployed configuration.

In some embodiments, when the occluder body is in the preliminary deployed configuration, the proximal tubular body segment extends proximally to the distal tubular body segment and/or forms with the distal tubular body segment a single layer extending between the occluder body proximal end and the occluder body distal end.

In some embodiments, when the occluder body is in the compressed deployed configuration, the proximal tubular body segment forces the distal tubular body segment to expand radially.

In some embodiments, when the occluder body changes from the preliminary deployed configuration to the compressed deployed configuration, the proximal tubular body segment is inserted into, and/or inwardly folded into, and/or tucked in, and/or stuffs, the distal tubular body segment, thereby forcing the distal tubular body segment to expand radially and/or to reshape elastically.

In some embodiments, the occluder body proximal end is distal to the proximal tubular body segment, when the occluder body is in the compressed deployed configuration.

In some embodiments, the proximal tubular body segment forms an inner layer relative to the distal tubular body segment, when the occluder body is in the compressed deployed configuration.

In some embodiments, the proximal tubular body segment is inverted when the occluder body changes from the preliminary deployed configuration to the compressed deployed configuration.

In some embodiments, the occluder body includes a portion between the occluder body proximal end and the distal tubular body segment comprising a crease and/or configured to facilitate a predetermined fold location and/or folding pattern when the proximal tubular body segment is pushed to advance towards the distal tubular body segment.

In some embodiments, the proximal tubular body segment is greater in average diameter than the distal tubular body segment, when the occluder body is in the preliminary deployed configuration.

In some embodiments, the proximal tubular body segment is greater in diameter than the distal tubular body segment along most or length thereof, when the occluder body is in the preliminary deployed configuration.

In some embodiments, a proximal portion of the proximal tubular body segment when the occluder body is in the preliminary deployed configuration, is configured to engage a distal portion of the distal tubular body segment when the occluder body is in the compressed deployed configuration.

In some embodiments, the occluder body reduces gradually in diameter in a direction from the occluder body proximal end to the occluder body distal end, when in the preliminary deployed configuration.

In some embodiments, at least part of the occluder body is cone-like shaped when in the preliminary deployed configuration and/or dome-like shaped when in the compressed deployed configuration.

In some embodiments, the occluder body covers entire length of the tension member when in the compressed deployed configuration.

In some embodiments, the tension member is fixated with a tension member distal end thereof to the occluder body distal end.

In some embodiments, the occluder body, when in the compressed deployed configuration, is configured to apply pressure to a wall portion enclosing a passage, thereby generating radial stress and/or friction sufficient for fixating the vascular occluder to the wall portion, wherein the passage is equal to or smaller in diameter than the occluder body when in the preliminary deployed configuration.

In some embodiments, a resistance to radial inward compression of the occluder body when changing from the preliminary deployed configuration to the compressed deployed configuration is increased by at least 10%.

In some embodiments, the tension member is rigid or flexible, and/or is configured as a rod or a wire.

In some embodiments, the vascular occluder further comprises a pulling member releasably connected or connectable to the tension member and configured to transfer a tension force from a proximal end thereof to the tension member. Optionally, the pulling member is flexible, and/or configured as a wire, and/or formed of Ni—Ti alloy. Optionally, the pulling member includes a connection element at a distal end thereof configured to selectively connect to or disconnect from a mating connection element provided at a proximal end of the tension member.

In some embodiments, the vascular occluder comprises an elongated pusher configured to transfer a pushing force via a proximal end thereof to the occluder body proximal end. Optionally, the pusher encloses a lumen extending along length thereof and sized for allowing unhindered travel of the pulling member therethrough. Optionally, the occluder body includes a force receiving surface located at an inner portion of the occluder body proximal end, configured to receive a pushing force from the pusher, the force receiving surface encloses an opening sized for allowing unhindered travel of the pulling member and/or the tension member therethrough.

In some embodiments, the vascular occluder further comprises a coupling portion connected to and/or extending from the occluder body proximal end, configured to connect to the pusher. Optionally, the puling member is configured to resist and/or prevent detachment of the coupling portion from the pusher when the pulling member extends taut along the coupling portion and the pusher. Optionally, the coupling portion includes a coupling portion cavity configured to receive a distal end portion of the pusher.

In some embodiments, the vascular occluder comprises a proximal locking portion connected to and/or extending from the occluder body proximal end, configured to engage a portion of the occluder body or the tension member, when the occluder body is in the compressed deployed configuration, and to resist axial extension of the occluder body from the compressed deployed configuration. Optionally, the proximal locking portion includes an inner surface configured to engage one or more motion resisting discrete recesses or protuberances emerging from a surface of tension member, and/or to lock to the tension member by way of friction with an outer surface of the tension member, so as to resist and/or prevent axial motion of the proximal locking portion relative to the tension member. Optionally, the proximal locking portion includes a front surface configured to engage by way of interlocking, bonding or clinging with a rear surface of a distal locking portion connected to and/or extending from the occluder body distal end, so as to lock the occluder body in the compressed deployed configuration. Optionally, the proximal locking portion includes an outer surface configured to engage by way of snugly fitting within, by way of friction with, and/or by way of clinging to, an inner surface of the occluder body distal end, so as to lock the occluder body in the chosen deployed configuration.

In some embodiments, the occluder body is elastically stretchable longitudinally into a delivery configuration having a delivery length and a delivery diameter, wherein the occluder body, when in the preliminary deployed configuration, has a preliminary deployed length smaller than the delivery length and a preliminary deployed diameter greater than the delivery diameter. Optionally, the occluder body, when in the compressed deployed configuration, has a compressed deployed length smaller than the preliminary deployed length and a compressed deployed diameter equal to or greater than the preliminary deployed diameter. Optionally, a ratio of maximal outer diameter to length of the occluder body is increased by at least 700% between the delivery configuration and the compressed deployed configuration. Optionally, a ratio of the delivery diameter to the delivery length is equal to or smaller than about 0.05 and the ratio of the compressed deployed diameter to the compressed deployed length is greater than about 0.5.

In some embodiments, the occluder body is at least partially meshed, woven and/or braided.

In some embodiments, the occluder body is at least partially impermeable and/or is covered or impregnated with an impermeable or hydrophilic substance or material.

In certain embodiments, there is provided a method which can comprise providing a vascular occluder comprising: an occluder body extending longitudinally between an occluder body distal end and an occluder body proximal end, the occluder body includes a proximal tubular body segment and a distal tubular body segment, and is elastically compressible longitudinally from a preliminary deployed configuration to a compressed deployed configuration; and a tension member. In some embodiments, the method includes releasing the vascular occluder in a passage having an inner diameter equal to or smaller than a compressed deployed diameter of the occluder body in the compressed deployed configuration. In some embodiments, the method includes allowing or causing the occluder body to form the preliminary deployed configuration, wherein the occluder body proximal end is not tethered to the occluder body distal end and/or the tension member is unconnected to the occluder body proximal end and/or to the occluder body distal end. In some embodiments, the method includes compressing the occluder body longitudinally until the occluder body is locked in the compressed deployed configuration, wherein the tension member tethers the occluder body proximal end to the occluder body distal end and held tightened therebetween.

In some embodiments, the compressing directly causes a local increase in radial stress in a wall portion forming the passage and/or a local increase in diameter of the passage.

In some embodiments, the compressing includes inverting the proximal tubular body segment.

In some embodiments, the proximal tubular body segment extends proximally to the distal tubular body segment when the occluder body is in the preliminary deployed configuration, wherein the compressing includes inserting the proximal tubular body segment inside the distal tubular body segment such that the distal tubular body segment is forced to expand radially and/or press radially against a wall portion forming the passage.

In some embodiments, the compressing includes reshaping the occluder body from a cone-like shape and/or to a dome-like shape.

In some embodiments, the releasing includes pushing the occluder body into the passage using a pusher, and/or by pulling the tension member using a pulling member connected thereto, wherein the compressing includes or is selectively followed by disconnecting the pulling member from the tension member.

In some embodiments, the method comprises pushing the occluder body through a catheter, when the occluder body is elastically stretched longitudinally in a delivery configuration having a delivery length and a delivery diameter, wherein the occluder body, when in the preliminary deployed configuration, has a preliminary deployed length smaller than the delivery length and a preliminary deployed diameter greater than the delivery diameter.

In some embodiments, the passage is formed by an artery in a live subject. In some embodiments, the releasing affects blood flow rate in the artery, and the compressing causes immediate or gradual reduction or blocking of the blood flow. In some embodiments, the releasing results in minimal or no anchoring of the occluder body to the artery, and the compressing results in or increases anchoring pressure applied by the occluder body to the artery.

In an aspect of some embodiments there is provided a kit comprising the vascular occluder and at least one of a catheter, an elongated pusher sized for extending along a lumen of the catheter, and a pulling member releasably connected or connectable to the tension member and sized for extending along a lumen of the pusher.

In certain embodiments, there is provided a vascular occluder comprising: an occluder body extending longitudinally between an occluder body distal end and an occluder body proximal end, the occluder body is elastically compressible and/or extendable longitudinally from an elastically relaxed configuration; a tension member fixated with a tension member distal end thereof to the occluder body distal end, and configured to facilitate longitudinal compression of the occluder body for occluding a bodily lumen portion, when the occluder body distal end is pulled with a tension member proximal end towards the occluder body proximal end; and a coupling portion connected to and/or extending from the occluder body proximal end, configured to releasably connect to a delivery system and/or to the tension member.

In some embodiments, the tension member is configured to resist and/or prevent releasing of the coupling portion from connecting with the delivery system and/or the tension member when the tension member extends along the coupling portion.

In some embodiments, the tension member includes a flexible wire.

In some embodiments, the delivery system includes a pusher configured to facilitate selective pushing of the vascular occluder into the bodily lumen portion, and the tension member is configured to resist and/or prevent detachment of the coupling portion from a pusher head of the pusher when the tension member extends taut along the coupling portion and the pusher head.

In some embodiments, the coupling portion is provided readily connected to the pusher head with the tension member passing throughout a passage enclosed by the coupling portion and/or by the pusher head, such that the tension member is confined to boundary of the passage.

In some embodiments, the vascular occluder is configured such that the tension member snugly fits and is unhandily movable axially in the passage.

In some embodiments, the coupling portion includes a coupling portion cavity opened laterally at a side of the coupling portion, configured to receive a laterally projecting portion of the pusher head configured to fit in the coupling portion cavity, wherein the tension member extends across the cavity and the laterally projecting portion so as to tether the laterally projecting portion to the coupling portion.

In some embodiments, the vascular occluder is configured such that the coupling portion and the tension member extend into a pusher head lumen via a pusher head opening in the pusher head, wherein the tension member extends in juxtaposition to a widening of the coupling portion in the pusher head lumen adjacent to the pusher head opening, thereby preventing passing of the widening through the pusher head opening, wherein the widening is sized to pass through the pusher head opening in absence of the tension member.

In some embodiments, the vascular occluder is configured such that the tension member passes through a coupling portion lumen extending along the coupling portion and protrudes laterally with a curved portion thereof via a coupling portion side opening on a lateral surface of the coupling portion to an opening area surrounded by a pusher head side opening on a lateral surface of the pusher head located immediately above the coupling portion side opening, thereby resisting or preventing longitudinal motion of the coupling portion relative to the pusher head.

In some embodiments, the coupling portion includes an actuator selectively actuatable from a normally opened configuration to a closed configuration, wherein the actuator is configured to facilitate unhindered axial motion of the tension member therethrough when in the closed configuration, and to engage the tension member when in the normally opened configuration and to lock the tension member to the coupling portion such that axial motion of the tension member therethrough is prevented.

In some embodiments, the actuator is actuatable from the normally opened configuration to the closed configuration when pressed distally under a pushing force sufficient to push the occluder body into the bodily lumen portion from a delivery catheter.

In some embodiments, the actuator includes a lever configured to shift the actuator from the normally opened configuration to the closed configuration when pressed to move from a less stressed position to a more stressed position.

In some embodiments, at least a proximal segment of the tension member is configured to break or disconnect, and/or be pulled away from the occluder body, when the tension member proximal end is pulled with a pulling force greater than a predetermined value.

In some embodiments, the vascular occluder is configured such that the coupling portion is allowed to detach from the delivery system or the tension member, when the at least proximal segment of the tension member is broken or disconnected, and/or pulled away, from the occluder body.

In some embodiments, the tension member is configured to divide into a tension member proximal segment and a tension member distal segment when the tension member proximal end is pulled with a pulling force greater than a predetermined value, such that the tension member distal segment is connected to the occluder body distal end and the tension member proximal segment is allowed to be withdrawn by pulling the tension member proximal end.

In some embodiments, the tension member includes a weakened portion and is configured to divide at or adjacent to the weakened portion when the tension member proximal end is pulled with a pulling force greater than a predetermined value.

In some embodiments, the weakened portion is located within, or distally to, the coupling portion, when the occluder body is in a chosen deployed configuration after being compressed axially from the elastically relaxed configuration.

In some embodiments, the vascular occluder comprising a locking feature comprising a proximal locking portion connected to and/or extending from the occluder body proximal end, configured to engage a portion of the occluder body or the tension member, when the occluder body is in a chosen deployed configuration after being compressed axially from the elastically relaxed configuration, and to resist axial extension of the occluder body from the chosen deployed configuration.

In some embodiments, the proximal locking portion includes an inner surface configured to engage one or more motion resisting discrete recesses or protuberances along a length of tension member, and/or to lock to the tension member by way of friction with an outer surface of the tension member, so as to resist and/or prevent axial motion of the proximal locking portion relative to the tension member.

In some embodiments, the proximal locking portion includes a front surface configured to engage by way of interlocking, bonding or clinging with a rear surface of a distal locking portion connected to and/or extending from the occluder body distal end, so as to lock the occluder body in the chosen deployed configuration.

In some embodiments, the proximal locking portion includes an outer surface configured to engage by way of snugly fitting within, by way of friction with, and/or by way of clinging to, an inner surface of the occluder body distal end, so as to lock the occluder body in the chosen deployed configuration.

In some embodiments, an occluding section of the occluder body, when in an elastically relaxed configuration, forms a tubular bellows-like shaped structure comprising a plurality of bellows sections.

In some embodiments, the occluder body is elastically stretchable longitudinally into a delivery configuration having a delivery length and a delivery diameter, and elastically compressible longitudinally into a deployed configuration having a deployed length smaller than the delivery length and a deployed diameter greater than the delivery diameter.

In some embodiments, the pusher includes a hollow tubular member. In some embodiments, the vascular occluder is configured such that the wire extends inside the hollow tubular member coaxially thereto. In some embodiments, the wire extends proximally at least to a proximal end of the hollow tubular member.

In certain embodiments, there is provided a method for occluding a bodily lumen portion, the method comprising: inserting a vascular occluder into the bodily lumen portion using a delivery system, the vascular occluder comprising an occluder body and a tension member; compressing the occluder body longitudinally to a chosen deployed configuration for occluding the bodily lumen portion; breaking or disconnecting at least a proximal segment of the tension member from the occluder body, thereby allowing release of the vascular occluder from the delivery system; and removing the delivery system

In some embodiments, the occluder body extends between an occluder body distal end and an occluder body proximal end, and the tension member is fixated with a tension member distal end thereof to the occluder body distal end, wherein the compressing includes pulling the occluder body distal end towards the occluder body proximal end by way of pulling the tension member.

In some embodiments, the occluder body includes or is connected to a coupling portion releasably connected to the delivery system and/or to the tension member, and the tension member extends taut along the coupling portion thereby resisting and/or preventing release of the coupling portion from the delivery system and/or the tension member, wherein the method includes releasing the coupling portion from the delivery system and/or the tension member.

In some embodiments, the breaking or disconnecting includes pulling the tension member with a force greater than a predetermined value and removing the at least proximal segment of the tension member away from the occluder body.

In some embodiments, the delivery system includes a catheter, and the inserting includes advancing the vascular occluder in a lumen of the catheter until the vascular occluder reaches the bodily lumen portion or adjacent thereto.

In some embodiments, the inserting includes withdrawing the catheter and/or pushing the vascular occluder with a pusher from within the lumen of the catheter such that most or all length of the occluder body protrudes outside the catheter in the bodily lumen portion or adjacent thereto.

In some embodiments, the compressing includes pulling the occluder body with the tension member against the catheter and/or the pusher.

In some embodiments, the coupling portion is releasably connected to a pusher head of the pusher and the tension member resists and/or prevents release of the coupling portion from the pusher head by extend along and engaging with the coupling portion and the pusher head, wherein the breaking or disconnecting includes disengaging and removing the at least proximal segment of the tension member from the coupling portion and the pusher head.

In some embodiments, the occluder body proximal end includes a proximal locking portion, and the compressing includes or is followed by engaging a portion of the occluder body or the tension member with the proximal locking portion so as to resist axial extension of the occluder body from the chosen deployed configuration.

All technical or/and scientific words, terms, or/and phrases, used herein have the same or similar meaning as commonly understood by one of ordinary skill in the art to which the invention pertains, unless otherwise specifically defined or stated herein. Illustrative embodiments of methods (steps, procedures), apparatuses (devices, systems, components thereof), equipment, and materials, illustratively described herein are exemplary and illustrative only and are not intended to be necessarily limiting. Although methods, apparatuses, equipment, and materials, equivalent or similar to those described herein can be used in practicing or/and testing embodiments of the invention, exemplary methods, apparatuses, equipment, and materials, are illustratively described below. In case of conflict, the patent specification, including definitions, will control.

### BRIEF DESCRIPTION OF THE DRAWINGS

Some embodiments are herein described, by way of example only, with reference to the accompanying drawings. With specific reference now to the drawings in detail, it is stressed that the particulars shown are by way of example and for purposes of illustrative description of some embodiments. In this regard, the description taken together with the accompanying drawings make apparent to those skilled in the art how some embodiments may be practiced.

In the drawings:

FIGS. 1A-1C schematically illustrate exemplary scenarios representing possible phases of blood flow occlusion following implantation of an exemplary vascular occluder in a target blood vessel, according to some embodiments;

FIGS. 2A-2C schematically illustrate exemplary scenarios representing steps in an exemplary procedure for implanting the exemplary vascular occluder of FIG. 1B, according to some embodiments;

FIGS. 3A-3B schematically illustrate an exemplary vascular occluder before and after compression, respectively, according to some embodiments;

FIGS. 4A-4C schematically illustrate exemplary scenarios representing steps in a method comprising releasing the exemplary vascular occluder of FIG. 3A from an exemplary delivery system, according to some embodiments;

FIGS. 5A-5B schematically illustrate the exemplary vascular occluder of FIG. 3A another exemplary locking feature before and after compression, respectively, according to some embodiments;

FIGS. 6A-6C schematically illustrate the exemplary vascular occluder of FIG. 3A with yet another exemplary locking feature before and after compression, respectively, according to some embodiments;

FIGS. 7A-7C schematically illustrate exemplary scenarios in an exemplary sequence for unlocking and releasing a coupling portion in an exemplary vascular occluder from a pusher head, according to some embodiments;

FIGS. 8A-8C schematically illustrate exemplary scenarios in another exemplary sequence for unlocking and releasing a coupling portion in another exemplary vascular occluder from a pusher head, according to some embodiments;

FIGS. 9A-9B schematically illustrate an exemplary locking feature, according to some embodiments;

FIGS. 10A-10D schematically illustrate exemplary scenarios in an exemplary sequence for releasing a coupling portion comprising an actuator in another exemplary vascular occluder, according to some embodiments;

FIGS. 11A-11B schematically illustrate respectively an exemplary vascular occluder with flow obstructing portions before and after axial compression thereof, according to some embodiments;

FIGS. 12A-12B schematically illustrate respectively an exemplary vascular occluder with flow obstructing portions before and after radial compression thereof, according to some embodiments;

FIGS. 13A-13B schematically illustrate respectively an exemplary vascular occluder with flow obstructing portions varying in radial location of less permeable region thereof, before and after axial compression thereof, according to some embodiments;

FIG. 14 schematically illustrates an exemplary vascular occluder comprising a plurality of bellows sections varying in diameter, according to some embodiments;

FIGS. 15A-15D schematically illustrate exemplary scenarios representing steps in an exemplary procedure for implanting the exemplary vascular occluder of FIG. 14, according to some embodiments;

FIGS. 16A-16B illustrate side views of an exemplary vascular occluder in a preliminary deployed configuration and in a compressed deployed configuration, respectively, according to some embodiments;

FIGS. 17A-17D illustrate side views of exemplary scenarios in an exemplary method for deploying the vascular occluder of FIG. 16 in a passage, according to some embodiments;

FIGS. 18A-18C illustrate side views of an exemplary compression mechanism of the vascular occluder of FIG. 16 in accordance with the exemplary method of FIGS. 17, according to some embodiments;

FIGS. 19A-19B illustrate cross-sectional side views of the exemplary compression mechanism of FIGS. 18A, according to some embodiments; and

FIGS. 20A-20B illustrate an exemplary release mechanism of a vascular occluder before and after activation thereof, respectively, according to some embodiments.

### DETAILED DESCRIPTION

Certain embodiments relate to devices and methods for treating a target blood vessel, and more particularly, but not exclusively, to vascular occluders and methods for deploying vascular occluders and/or occluding target blood vessels, such as arteries or veins.

In some embodiments, there is provided a vascular occluder optionally intended for use similarly to a vascular plug or another type of mechanical embolization device. The vascular occluder includes an elongated occluder body optionally formed at least in part from a braided sleeve-like structure and is selectively compressible along longitudinal axis thereof from a stretched delivery length and form to a final and/or chosen compressed deployed length and form. The vascular occluder can be left in a chosen target endoluminal portion of a blood vessel (e.g., artery) when it is fixated in the deployed length.

Compression of the vascular occluder can be performed with and/or via a delivery catheter with other delivery elements that can be used to eject the vascular occluder and/or manually compress it in the blood vessel. Compression can be achieved by moving distal and proximal body ends of the occluder body towards each other along the longitudinal axis such as by pressing (e.g., squeezing) internal portion of the distal and/or proximal body end in proximity to the longitudinal axis while allowing more peripheral (outer) portions of the distal and/or proximal body end to remain more loose.

The occluder body may include an occluding section, optionally configured as a bellows-like structure, extending longitudinally between the distal body end and the proximal body end. The bellows-like structure includes a plurality of tubular folds, each fold comprising at least one flow obstructing portion configured to limit or block blood from flowing therethrough. When the occluder body is compressed into the deployed length, the flow obstructing portions extend transversally, or near transversally to the longitudinal axis for obstructing blood flow along the longitudinal axis. Furthermore, the radially inner portions of the folds, which are fixedly pressed tight against each other, are parallel to the longitudinal axis thereby further increasing the obstruction to flow while causing increase in the radial force transferred to the blood vessel wall which anchors the vascular occluder thereto.

In some embodiments, the vascular occluder is fixedly deformable into the compressed deployed length configuration as described, optionally by use of a structural element (e.g., a tension member) configured to lock and/or continuously (e.g., permanently) pull the radially inner portions of the folds and/or distal and proximal body ends of the occluder body towards each other along the longitudinal axis. This structural element may be configured as part of locking mechanism which fastens the radially inner portions together after the operator causes compression therebetween. Optionally, additionally or alternatively, the structural element may be configured as a plastically deformable member (e.g., in a form of one or more plastically deformable wires braided into the occluder body and is mechanically capable of withstanding inner and/or outer stresses optionally acting to re-elongate the occluder body from the compressed length configuration). Optionally, additionally or alternatively, the structural element may be configured as an elastically deformable member optionally connecting between the radially inner portions of the distal and proximal body ends across the folds and being in a relaxed or in elastically stretched form when the occluder body is in the compressed length, thereby resisting longitudinal extension and/or applying continuous pulling force therebetween parallel to the longitudinal axis.

The structural and/or functional features described above allow creation of a vascular occluder capable of substantially greater ratios of outer dimensions from stretched delivery length to final compressed length, thereby allowing use of smaller microcatheters with outer diameter of 2.8 Fr or less, for example, for treating small to medium or even large arteries greater than about 5 mm, for example. The use of a structural element, such as a tension member, for locking and/or continuously pulling together radially inner portions of the occluder body's folds and/or proximal and distal body ends improves anchoring to the blood vessel wall for diminishing or preventing migration potential by generating increased radially pressure by outward or radially outer portions of the occluder body to the blood vessel wall resulting from the continuous pulling force of structural element on the occluder body radially inner portions. The unique occluder compression presented also facilitates a more focal or accurate placement of the vascular occluder in a smaller length portion of the blood vessel, thereby improving fitting and positioning for different occlusion requirements, which may be considered advantageous in different scenarios for example in cases of arterial raptures or pseudoaneurysms, in which the treatment often requires a focal occlusion of the vessel on both sides of the rapture.

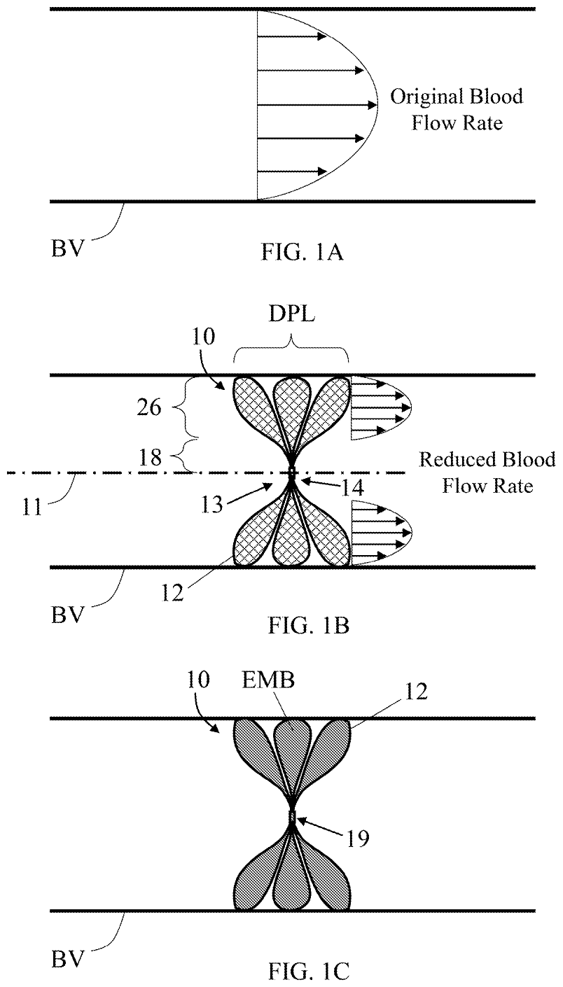

FIGS. 1A-1C schematically illustrate exemplary scenarios representing possible phases of blood flow occlusion following implantation of an exemplary vascular occluder 10 (shown in a side cross-sectional view) in a target blood vessel BV. FIG. 1A shows target blood vessel BV (e.g., artery) before catheter insertion and embolization. FIG. 1B shows same portion of blood vessel BV partially occluded with vascular access port 10 that is in a final compressed deployed length DPL configuration immediately following deployment and catheter withdrawal from the body. FIG. 1C shows same portion of blood vessel BV with vascular occluder 10 following implantation, after it had absorbed and/or accumulated clots EBM sufficiently to diminish substantially or completely block blood flow therethrough. Optionally, additionally or alternatively, some or most of blood vessel BV can be clotted following days to months from implantation, besides vascular occluder 10 as shown, following sufficient clot accumulation in vascular occluder 10.

Vascular occluder 10 is shown as having a meshed (e.g., braided) occluder body 12 that is optionally configured also for allowing accumulation of clots and/or emboli therein and/or in mesh openings thereof, however occluder body 10 may alternatively include, instead or over meshed structure thereof, a fluid tight material (e.g., membrane) impermeable to blood flowing therethrough, for causing immediate occlusion once deployed with no need for clots and/or emboli accumulation. Vascular occluder 10 is shown in a symmetric configuration relative to a longitudinal axis 11 thereof for illustrative purpose also for demonstrating the significance of maintaining (e.g., locking) it under a continuous (e.g., permanent) compression particularly across radially inner portions thereof from both ends thereof, however its outward or radially outer portions, located farther to longitudinal axis 11 than the compressed radially inner portions, can be more loose and allowed to deform during or after deployment in same or any other, optionally unsymmetrical, configuration.

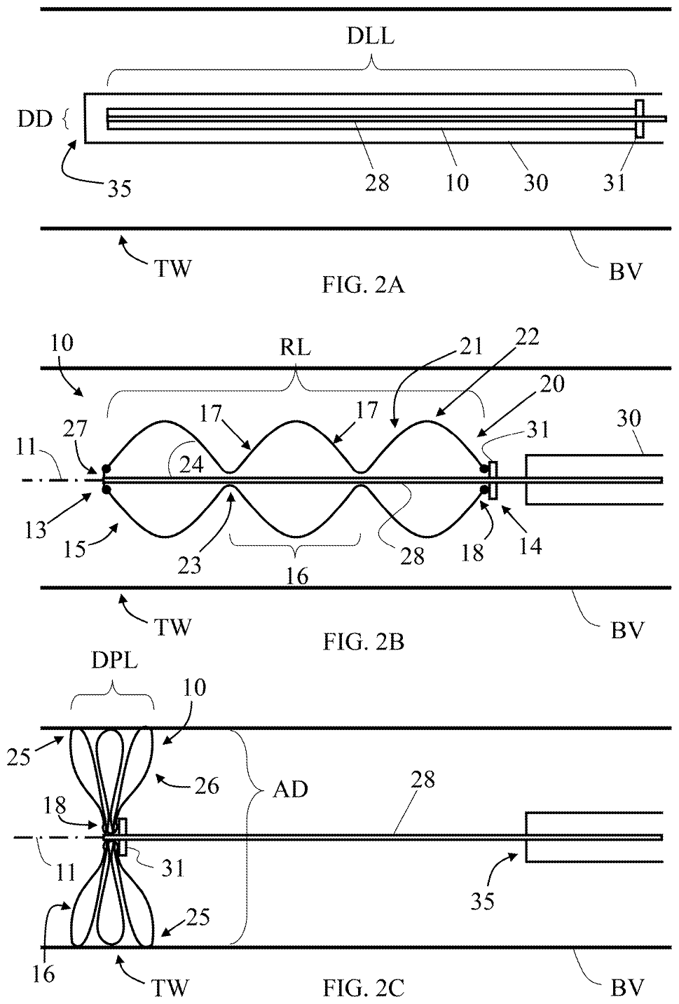

Occluder body 12 has generally elongated tubular shape when in a relaxed (i.e., not elastically stressed) length RL, as shown in FIG. 2B for example, and is comprising a distal body end 13, a proximal body end 14 and an occluding section 15, that is configured as a bellows-like structure, extending between distal body end 13 and proximal body end 14, and longitudinally compressible along longitudinal axis 11. A tension member 28 extends along occluding section 15 and connected with a distal end thereof to distal end of occluding section 15 (e.g., optionally same as distal body end 13, as shown in the figures). Tension member 28 is configured to facilitate selective longitudinal compression of the occluding section 15 by applying tension to the distal end of occluding section 15 via tension member 28 while pushing distally a proximal end of occluding section 15 (e.g., optionally same as proximal body end 14, as shown in the figures). Occluder body 12 is longitudinally stretchable to delivery length DLL wherein the occluding section 15 is configured for passing unhinderedly through a microcatheter into target blood vessel BV. Occluder body 12 is also longitudinally compressible to deployed length DPL thereby radially extending with one or more anchoring portions thereof to a deployed diameter DD greater than diameter of the target blood vessel BV, wherein the occluding section 15 is configured to limit or block blood from flowing therethrough.

In some embodiments, the deployed length DPL is about 50% or less than the delivery length DLL. In some embodiments, the delivery length DLL (when residing stretched in a microcatheter, for example) is greater than about 10 mm, optionally greater than about 20 mm, and the deployed length DPL is smaller than about 6 mm, optionally smaller than about 3 mm. The maximal outer diameter of occluder body 12 is smaller than about 2 mm, optionally smaller than about 1.5 mm, when occluder body 12 is in the delivery length DLL and greater than about 3 mm, optionally greater than about 5 mm, when occluder body 12 is in the deployed length DPL. The ratio of maximal outer diameter to length of the occluder body 12 is optionally smaller than about 0.05 when occluder body 12 is in the delivery length DLL and greater than about 0.5 when occluder body 12 is in the deployed length DPL.

Occluding section 15 includes a plurality of tubular folds 16, each fold 16 comprising at least one flow obstructing portion 17 configured to limit or block blood from flowing therethrough. Occluder body 12 is selectively and fixedly compressible between distal body end 13 and proximal body end 14 parallel to longitudinal axis 11, from being stretched in delivery length DLL (as shown in FIG. 2A, for example) wherein the flow obstructing portions 17 extend substantially parallel to longitudinal axis 11, to being compressed in deployed length DPL (as shown in FIG. 1B and FIG. 2C, for example) wherein the flow obstructing portions extend transversally to longitudinal axis 11. As shown, occluder body 12 is configured, when in the deployed length DPL, such that radially inner portions 18 of the plurality of tubular folds 16 and/or of distal and proximal body ends 13 and 14 are fixedly pressed tight against each other parallel to longitudinal axis 11. Occluder body 12 is optionally formed of a braided or interwoven sleeve interbraided from at least one wire and configured to expand in diameter when compressed longitudinally.

Occluder body 12 encloses a lumen 27 that extends along longitudinal axis 11 and across the flow obstruction portions 17 also when occluder body 12 is compressed in its final deployed length DPL configuration. Tension member 28 may be provided integrated in vascular occluder 10 or as a separate member, in a kit for example. Tension member 28 is configured to pass through lumen 27 and to be fixedly connected to at least one of the body ends 13 and 14 or of one the flow obstruction portions 17, such that the occluder body 12 is longitudinally compressible from the delivery length DLL to the deployed length DPL along the elongated tension member 28 which is optionally coinciding with longitudinal axis 11.

Tension member 28 may be configured as a length securing member configured to resist lengthwise changes from the deployed length of occluder body 12. As such, tension member 28 may be part of a locking mechanism 19 and provided at one end of the occluder body 12 and configured to engage and selectively connect to a mating portion or member provided at another end of the occluder body, when occluder body 12 is in the deployed length DPL. Tension member 28 may be configured as a plastically deformable element or an elastically deformable element, optionally in a form of a wire or coil. Tension member 28 may be configured to plastically deform when occluder body 12 is compressed longitudinally from the delivery length DLL to the deployed length DPL and/or when the occluder body 12 is forced to change in length from the deployed length DPL. Tension member 28 may otherwise be configured to elastically relax (optionally not to a fully relaxed state) when occluder body 12 is compressed longitudinally from the delivery length DLL to the deployed length DPL and/or to elastically resist a change in length of occluder body 12 from the deployed length DPL.

Each one of the tubular folds 16 includes a pair of adjacent ascending portion 20 and descending portion 21, each of them extending between respective outward apex 22 projecting away from longitudinal axis 11 and inward crease 23 projecting towards longitudinal axis 11. Each ascending portion 20 and/or each descending portion 21 includes one of the flow obstructing portions 17 between respective apex 22 and crease 23. A fold angle 24 formed between longitudinal axis 11 and the ascending or descending portion in each pair 16 is smaller than about 10°, optionally smaller than about 5°, when occluder body 12 is in the delivery length DLL and greater than about 15°, optionally greater than about 45°, optionally greater than about 80°, when occluder body 12 is in the deployed length DPL.

Each one of tubular folds includes an anchoring portion 25 adjacent to the respective outward apex 22 thereof. When occluder body 12 compresses, a plurality of anchoring portions 25 are forced to expand in diameter sufficiently for engaging with and/or pressing against a target wall portion of a blood vessel, laterally outwardly to longitudinal axis 11, for fixating flow obstructing portions 17 to the target wall portion. The vascular occluder 10 is configured such that a plurality of the flow obstructing portions 17 are distant with each other when occluder body 12 is in the delivery length DLL and are compressed against each other when occluder body 12 is in the deployed length DPL. The plurality of the flow obstructing portions 17 is configured to obstruct blood flow therethrough when occluder body 12 is in the deployed length DPL or to allow a smaller rate of blood flowing therethrough than when occluder body 12 is in the delivery length DLL.

In some embodiments, in each fold 16, the respective radially inner portion 18 is located closer to the respective inward crease 23 than to the respective outward apex 22. In some embodiments, when occluder body 12 is in the deployed length DPL, the radially inner portions 18 of the plurality of folds 16 are deformed into a condensed form with no gap therebetween, and as such they may be configured to block blood flow therethrough, optionally allowing flow through radially outer portions 26 (peripheral or outward portions of folds 16 spaced radially outwardly from the radially inner portions 18) around it as shown in FIG. 1B for example. The radially outer portions 26 of folds 16 are also allowed to shift relatively to each other and/or to be arranged with gaps therebetween. Therefore, occluder body 12 in the deployed length DPL is thinnest adjacent to the radially inner portions 18 and increasing gradually in thickness radially outwardly to radially inner portions 18.

As described above, the elongated occluder body 12 is deliverable via the lumen of a microcatheter when stretched into the delivery length DLL, where it is configured in a cylindrical-like form having a delivery diameter DD substantially constant along longitudinal axis 11. When in deployed length DPL, occluder body 12 is configured such that each one of tubular folds 16 is expandable to an anchoring diameter AD adjacently to the outward apex 22 thereof, measurable when in relaxed length RL. When expanded to anchoring diameter AD, that is substantially greater than inner diameter of the blood vessel at the implantation site, occluder body 12 is configured to apply an anchoring force laterally outwardly to longitudinal axis 11 for fixation to the target wall portion, being sufficient for facilitating engaging and pressing against a target wall portion of blood vessel BV. The anchoring force is optionally equal to or greater than 400 gr. Delivery diameter DD is optionally equal to or smaller than about 1.5 mm, optionally particularly equal to or smaller than about 1 mm, optionally particularly equal to or smaller than about 0.5 mm. Anchoring diameter AD is optionally equal to or greater than about 3 mm, optionally particularly equal to or greater than about 5 mm, optionally equal to or greater than about 7 mm, or optionally equal to or greater than about 12 mm.

As will be described in more detail with respect to FIGS. 2 to 4, methods for occluding blood vessel BV can begin with inserting vascular occluder 10 into the blood vessel proximately to a target wall portion TW of blood vessel BV using a microcatheter 30. Microcatheter 30 may be designated for angiography and/or embolization of peripheral blood vessels, optionally having inner lumen diameter between about 0.45 mm and about 0.7 mm, and length between about 100 cm and 150 cm, for example. In some other embodiments, microcatheter 30 diameter is greater than 0.7, and is optionally between about 1 mm and about 2 mm. In microcatheter 30, vascular occluder 10 is maintained stretched from its relaxed length RL in delivery length DLL. Once in proper position, vascular occluder 10 can be completely ejected from microcatheter 30, optionally using a manually operable pusher 31, thereby optionally regaining its relaxed length RL or closer thereto. Vascular occluder 10 can then be initially compressed longitudinally, such that the plurality of tubular folds 16 with flow obstruction portions 17 thereof become condensed and being adjacent and substantially parallel to each other.

Following the initial compression, the operator (e.g., physician) can cause the final compression (e.g., tightening) required for putting occluder body 12 in the fixed deployed length DPL. The operator can apply pusher 31 or distal end of microcatheter 30, which are sized and configured to engage only radially inner portions 18, for pressing radially inner portions 18 tight against each other, parallelly to longitudinal axis 11, such that radially inner portions 18 are deformed into a condensed form, optionally to a full extent with no gap therebetween. By pressing tight only radially inner portions 18, the radially outer portions 26 of the plurality of folds 16 are allowed to shift relative to each other and/or to arrange with gaps therebetween, either autonomously or in response to external forces acting thereon such as from the target wall portion TW.

As part of compressing occluder body 12 or upon tightening together radially inner portions 18, the flow obstructing portions 17 are subjected to a radial force being sufficient to fixate vascular occluder 10 to the target wall portion TW with by engaging directly with outward apexes 22 of tubular folds 16. The vascular occluder insertion may include positioning distal body end 13 or one or more (e.g., distal-most) of flow obstructing portions 17 adjacent to the target wall portion TW and the fixating includes pushing distal body end 14 and/or a proximal-most one of the flow obstructing portions 17 towards the distal body end 13 or the distal-most flow obstructing portion.

FIGS. 2A-2C schematically illustrate exemplary scenarios representing steps in an exemplary procedure for implanting vascular occluder 10 for occluding blood vessel BV. FIG. 2A shows a first exemplary scenario in which microcatheter 30 is advanced distally in blood vessel BV until reaching adjacently to target wall portion TW with a distal end 35 of microcatheter 30. Vascular occluder 10 may be readily provided in microcatheter 30 or advanced distally therein after microcatheter 30 is in blood vessel BV and/or distal end 35 is adjacent to target wall portion TW. When residing in microcatheter 30, occluder body 12 is stretched in delivery length DLL configuration for allowing unhindered insertion into and passing through microcatheter 30. Elongated tension member 28 is fixated with distal end thereof to distal body end 13 of occluder body 12, with proximal end thereof is connected to a portion of microcatheter 30 or to another element in microcatheter 30, or is provided free and optionally extending out of patient's body. Pusher 31 is located in microcatheter 30 proximally to vascular occluder 10 and is configured for sliding over tension member 28 when stretched to coincide substantially with longitudinal axis 11.

FIG. 2B shows a second exemplary scenario, after vascular occluder 10 has been completely ejected from microcatheter 30 within blood vessel BV, such that distal body end 13 of occluder body 12 is positioned adjacent to target wall portion TW. In some embodiments, this can be accomplished, such as following the first exemplary scenario in FIG. 2A, by holding pusher 31 in place or pushing it distally, while pulling proximally microcatheter 30 until it is completely withdrawn relatively to vascular occluder 10 as shown. In this scenario, occluder body 12 may be in relaxed length RL configuration, as shown, or is partially compressed between pusher 31 and distal body end 13 (which may be held in place or pulled proximally with tension member 28) to a length between relaxed length RL and deployed length DPL.

FIG. 2C shows a third exemplary scenario after vascular body 12 is compressed into deployed length DPL configuration. As shown and as previously described, compression is made to a greater extent closer to longitudinal axis 11 at radially inner portions 18 of folds 16, making occluder body 12 substantially thinner in proximity to longitudinal axis 11 relative to radially outer portions 26 of folds 16. This configuration can be accomplished by pushing pusher 31 against radially inner portions 18 while holding distal body portion 13 of occluder body 12 in place (adjacent to target wall portion TW) using tension member 28 connected thereto. Occluder body 12 is configured to maintain is deployed length DPL even if pusher 31 is withdrawn, optionally by use of tension member 28 shown in FIG. 1C. In some embodiments, distal portion or entire length of tension member 28 is configured as a length securing member, and it is applied to resist or prevent extension of occluder body from deployed length DPL. The operator may relax and redeploy vascular occluder 10 as needed, for example for repositioning. Once correctly implanted and anchored adjacent to target wall portion TW in blood vessel BV, microcatheter 30 and pusher 31 can be withdrawn, and tension member 28 can also be withdrawn, left implanted as part of vascular occluder 10, or cut in length leaving distal portion thereof connected to and implanted with vascular occluder 10.

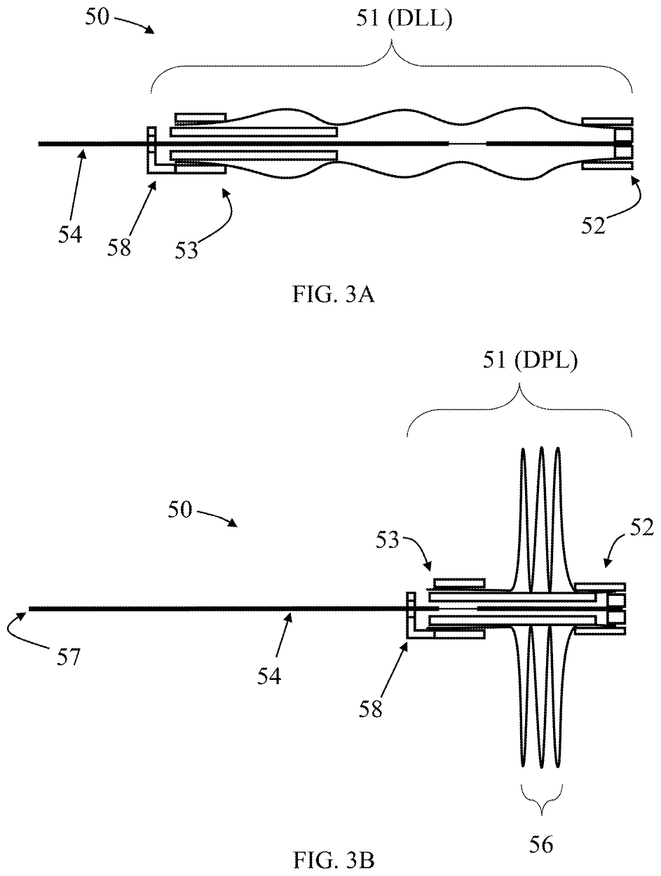

FIGS. 3A-3B schematically illustrate a vascular occluder 50 before and after compression, respectively. Vascular occluder 50 may be similar or identical to vascular occluder 10 at least in part, optionally as an exemplary variation thereof, and/or may include one or more similar or identical structural and/or functional feature described above. Vascular occluder 50 includes an occluder body 51 extending longitudinally between an occluder body distal end 52 and an occluder body proximal end 53. Occluder body 51 is elastically extendable longitudinally from an elastically relaxed configuration, and is elastically compressible from the elastically relaxed configuration to a chosen deployed configuration. In its elastically relaxed configuration, optionally similarly or identically to occluder body 12 (as shown in FIG. 2B, for example), an occluding section 56 of occluder body 51 forms a tubular bellows-like shaped structure comprising a plurality of bellows sections. Occluder body 51 is elastically stretchable longitudinally into a delivery configuration (as shown in FIG. 3A) having a delivery length DLL and a delivery diameter, and elastically compressible longitudinally into the chosen deployed configuration (as shown in FIG. 3B) having a deployed length DPL smaller than delivery length DLL and a deployed diameter greater than the delivery diameter.

Vascular occluder 50 also includes a tension member 54 fixated with a tension member distal end thereof to occluder body distal end 52. Tension member 54 may include, be configured as, or formed with, a flexible non-extendable wire, optionally made of nylon, stainless steel or Ni—Ti alloy, and is configured to facilitate longitudinal compression of occluder body 51, by pulling tension member proximal end 57 thereof, optionally from outside patient's body. For example, occluder body distal end 52 can pulled towards occluder body proximal end 53 with tension member 54 while occluder body proximal end 53 is held in-place or pushed such as by using a delivery system (e.g., a catheter and/or a pusher). Otherwise, occluder body proximal end 53 may be pushed towards occluder body distal end 52 using a delivery system while occluder body distal end 52 is held in-place or pulled with tension member 54.

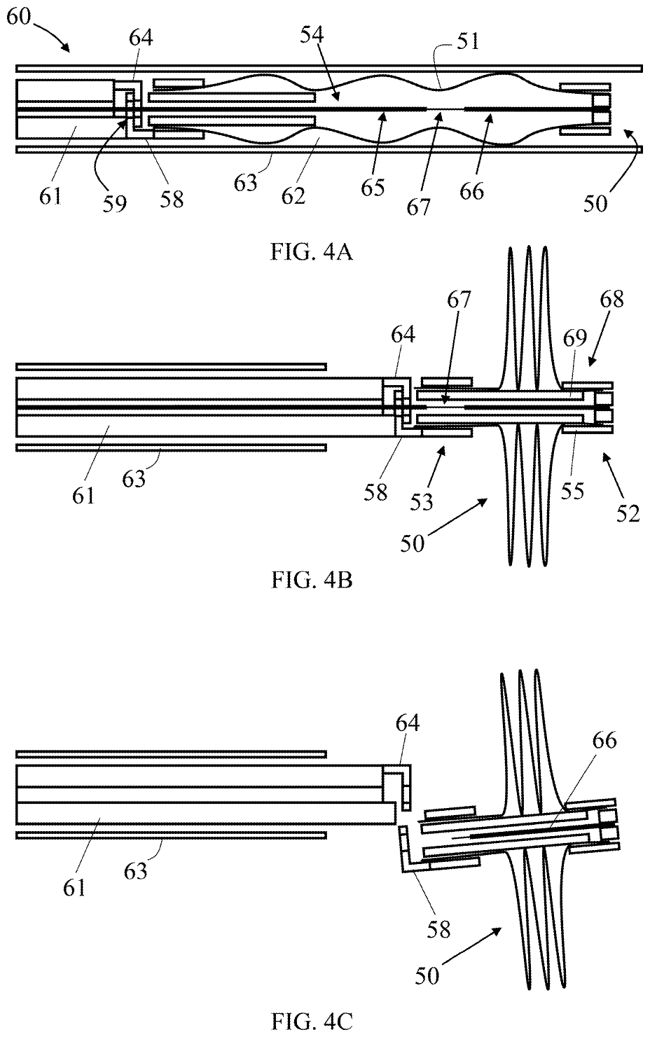

Vascular occluder 50 also includes a coupling portion 58 connected to and/or extending from occluder body proximal end 53 and configured to releasably connect to a delivery system. Tension member 54 extends along coupling portion 58 and therefore resists and/or prevents releasing of coupling portion 58 from connecting with the delivery system, as will be described in more details hereinafter. FIGS. 4A-4C schematically illustrate exemplary scenarios representing steps in a method wherein vascular occluder 50 is first held locked (tethered) to a delivery system 60, and then released therefrom after being compressed to a chosen deployed configuration. Delivery system 60 includes a pusher 61 configured to facilitate selective pushing of vascular occluder 50 body, optionally from a lumen 62 of a catheter 63 (which may also be part of delivery system 60), into the target bodily lumen portion. Coupling portion 58 is optionally provided readily connected to a pusher head 64 provided at a front (distal) end of pusher 61. Tension member 54 extends throughout and confined to a passage 59 enclosed (sequentially) by coupling portion 58 and pusher head 64, and therefore resists and/or prevents detachment of coupling portion 58 from pusher head 64. Optionally, passage 59 as a minimal or constant inner diameter similar in size to outer diameter of tension member 54, and optionally the tension member 54 snugly fits in passage 59.

Tension member 54 is fixedly connected with distal end thereof to occluder body distal end 52. Fixation of tension member 54 to occluder body distal end 52 can be achieved by one or more fixations means or processes, such as for example by way of bonding, welding, friction, crimping, plastic deformation, and/or clamping, made between portions of occluder body distal end 52, and/or between members or layers forming it. In an example, occluder body distal end 52 includes an outer tube enclosing a braided member portion, and is connected to tension member 54 by way of crimping the outer tube until it is plastically yields and presses tight over and against the braided member portion and the portion of tension member 54 passing therethrough. In some embodiments, fixation of tension member 54 is configured to withstand detachment from occluder body distal end 52 under pulling forces smaller than about 100 N (Newton), optionally smaller than about 10 N, optionally smaller than about 5 N, optionally smaller than about 3 N.

In some embodiments, vascular occluder 50 is configured such that tension member 54 is releasable (such as by way of disconnection, decoupling, or detachment, for example) from connection to occluder body distal end 52 when under tension generated with a pulling force greater than a predetermined value, optionally greater than about 0.5 N, optionally greater than about 1 N, optionally greater than about 2 N, or optionally greater than about 3 N, for example. In some such embodiments, tension member 54 is releasably connected to occluder body distal end 52 with a widening (in a portion thereof, or with a wider member connected thereto, for example) that resists detachment of tension member 54 when under tension smaller than the predetermined value, and/or tension member 54 is held with a static friction force smaller than the said predetermined value.

Alternatively or additionally, and as shown, tension member 54 is configured to break (e.g., divide) into a proximal segment 65 and a distal segment 66 when under tension generated with a pulling force greater than a predetermined value, optionally greater than about 0.5 N, optionally greater than about 1 N, optionally greater than about 2 N, or optionally greater than about 3 N, for example. Once broken, proximal segment 65 of tension member 54 can be pulled away from the occluder body, leaving distal segment 66 connected to occluder body distal end 52 and in occluder body 51. Tension member 54 includes a weakened portion 67 and it is configured to divide at or adjacent to weakened portion 67 when tension member proximal end 57 is pulled with a pulling force greater than the predetermined value. Vascular occluder 50 is optionally configured such that weakened portion 67 is located within or distally to coupling portion 58, when occluder body 51 is in the chosen deployed configuration. When proximal segment 65 of tension member 54 is broken and pulled away from occluder body 51, coupling portion 58 is no longer locked (tethered) to pusher head 64 and therefore it can freely or be selectively shifted to detach from pusher head 64.

Vascular occluder 50 includes a locking feature 68 configured to lock occluder body 51 in the chosen deployed configuration after being compressed longitudinally from the elastically relaxed configuration and to resist axial extension of occluder body 51 from the chosen deployed configuration. Locking feature 68 optionally comprises a proximal locking portion 69 connected to and/or extending distally from occluder body proximal end 53, and a distal locking portion 55 connected to and/or located in occluder body distal end 52. Proximal locking portion 69 is configured to engage with an outer surface thereof to an inner surface of distal locking portion 55 by snugly fitting and/or way of friction so as to lock occluder body 51 in the chosen deployed configuration.

Using vascular occluder 50 with delivery system 60 can be applied in different methods for achieving different end results, such as for example for occluding a blood vessel, for dilating a portion in a blood vessel, for removing clots, or others. For occluding a body lumen portion, such as of an artery or a vein, in a body of a live subject, vascular occluder 50 can be inserted into the bodily lumen portion using delivery system 60, wherein delivery system can include pusher 61 and/or catheter 63. For delivery of vascular occluder 50, catheter 63 may first be extended along a chosen route in subject's vasculature such that a proximal end thereof is accessible from outside subject's body and a distal end thereof is positioned at or in proximity to the target bodily lumen portion. Vascular occluder 50 can be already provided in lumen 62 of catheter 63 or inserted into and through lumen 62 only after proper positioning of catheter 63. Delivery of vascular occluder 50 via lumen 62 can be accomplished when occluder body 51 is stretched from its elastically relaxed configuration, as shown in FIG. 4A for example. Alternatively, vascular occluder 50 can be delivered without use of a catheter and be pushed directly within bodily vasculature.

Occluder 50 may be provided already connected to pusher 61 or the operator may connect them together or leave them unconnected, before inserting vascular occluder in lumen 62, for pushing it through catheter 63, and for deployment thereof. Connecting together pusher 61 and vascular occluder 50 may be considered or prescribed as a recommended or a necessary precondition for applying the method of treatment, or for one or more aspects thereof, such for safety consideration or for the possibility to remove or reposition vascular occluder 50 in different phases of the method, during delivery or deployment, such as after compression and/or locking occluder body 51 in the chosen deployed configuration and before tension member 54 is broken, disconnected or detached from occluder body 51. As described above, pusher 61 and vascular occluder 50 can be connected between pusher head 64 and coupling portion 58, with tension member 54 functioning as a safety or security element to lock or tether them together so they cannot disconnect, uncouple and/or disengage from each other as long as tension member 54 is intact and extends therethrough.

When in the bodily lumen portion, occluder body 51 can be compressed longitudinally to a chosen deployed configuration for occluding the bodily lumen portion, as shown in FIG. 4B, similarly or identically to as described with respect to vascular occluder 10, to FIGS. 1 and/or to FIG. 2. For example, catheter 63 can be at least partly withdrawn and/or vascular occluder 50 can be pushed with pusher 61 from within catheter lumen 62 such that most or all length of occluder body 51 protrudes outside catheter 63 in the bodily lumen portion or adjacent thereto, and then occluder body 51 can be compressed by pulling occluder body 51 with tension member 54 against catheter 63 and/or pusher 61. Compressing occluder body 51 to the chosen deployed configuration optionally includes or is followed by engaging a portion of occluder body 51, optionally particularly distal locking portion 55, with proximal locking portion 69 so as to resist axial extension of occluder body 51 from the chosen deployed configuration.

In order to release vascular occluder 50 from delivery system 60, and particularly from pusher 61, tension member 54 is pulled with a force greater than a predetermined value until proximal segment 65 is broken or otherwise divided from distal segment 66 of tension member 54 at or adjacent to weakened portion 67, and proximal segment 65 is then removed away from occluder body 51 and particularly from passage 59. As shown in FIG. 4C, coupling portion 58 can then be easily released from connecting and/or mechanically engaging to pusher head 64, either by inertial or auxiliary forces, or by selective manipulation of the operator. Delivery system 60, including catheter 63 and pusher 61, can be withdrawn and removed from subject's vasculature.

Locking feature 68 of vascular occluder 50 is designed such that an element or an extension of occluder body proximal end 53, comprising proximal locking portion 69, extends distally and is configured with mechanical properties, size and shape to lock (e.g., by way of static fiction, for example) with an inner surface of distal locking portion 55. Distal locking portion 55 may be a portion of a rigid element having a friction coefficient and contact surface for developing sufficient static friction force capable of holding firmly proximal locking portion 68 after occluder body 51 is compressed in the chosen deployed configuration. Alternatively or additionally, distal locking portion 55 may include a meshed (e.g., woven or braided) and/or textured material, optionally a portion of the tubular bellows-like shaped structure fixated occluder body distal end 52, and proximal locking portion 69 can be configured to connect thereto by way of friction or by way of hooking to its mesh openings or textured surface, for example.

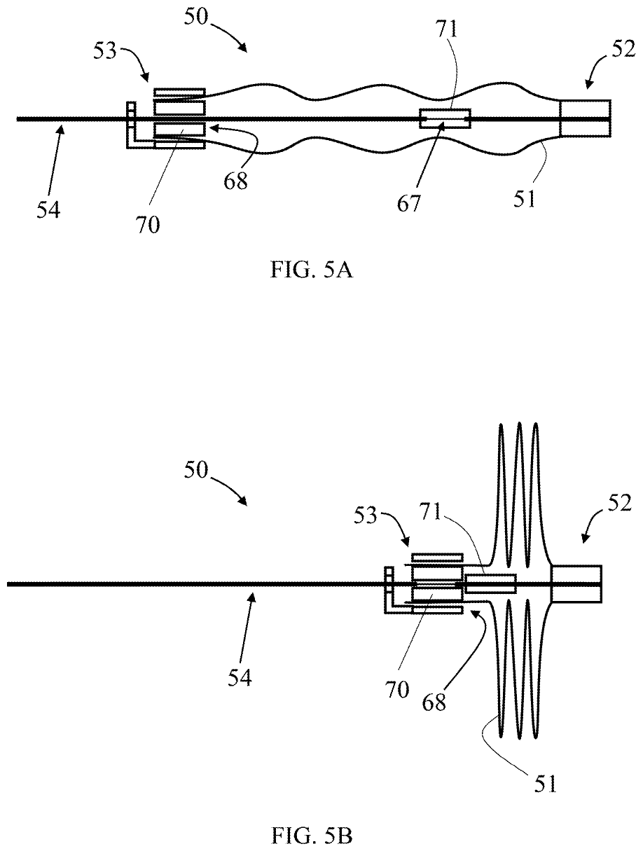

FIGS. 5A-5B schematically illustrate vascular occluder 50 with a different configuration of locking feature 68 before and after compression, respectively. Other functional and structural features may be similar or identical to as described with respect to FIGS. 3 and 4. In this exemplary embodiment, locking feature 68 includes a proximal locking portion 70 connected to and/or extending from occluder body proximal end 53, and configured to engage a portion of tension member 54 for locking occluder body 51 in a chosen length and/or to resist re-extension thereof. In some embodiments, proximal locking portion 68 is configured to maintain a continuous or discrete contact with a surface area of tension member 54 such that it resists motion of tension member 54 (e.g., by way of friction and/or by way of engaging one or more motion resisting discrete recesses or protuberances along a length of tension member 54, for example) therethrough under extension forces acting on occluder body 51, and optionally allows motion of tension member 54 therethrough when it is pulled with a pulling force greater than a predetermined value, optionally greater than about 0.3 N, optionally greater than about 0.5 N, or optionally greater than 1 N, for example. As such, the user can have an option to choose compression length during deployment as the chosen compressed configuration rather than operate locking feature 68 to lock occluder body 51 in a predetermined deployed configuration.

In some embodiments, tension member 54 and/or proximal locking portion 70 is configured such that sufficient and/or maximal locking force is achieved only when occluder body 51 is compressed to the chosen (predetermined) deployed configuration, and for example tension member 54 may have one or more covered, widened and/or textured portions at a specific location along its length so that they are engaged with proximal locking portion 70 only when occluder body 51 is in the chosen deployed configuration. In some embodiments, weakened portion 67 is located within or adjacent to proximal locking portion 70 when occluder body 51 is in the chosen deployed configuration. In some embodiments, weakened portion 67 is covered with a covering 71, and optionally covering 71 is be engaged with proximal locking portion 70, and/or be pushed aside to uncover weakened portion 67, when occluder body 51 is compressed to the chosen deployed configuration.

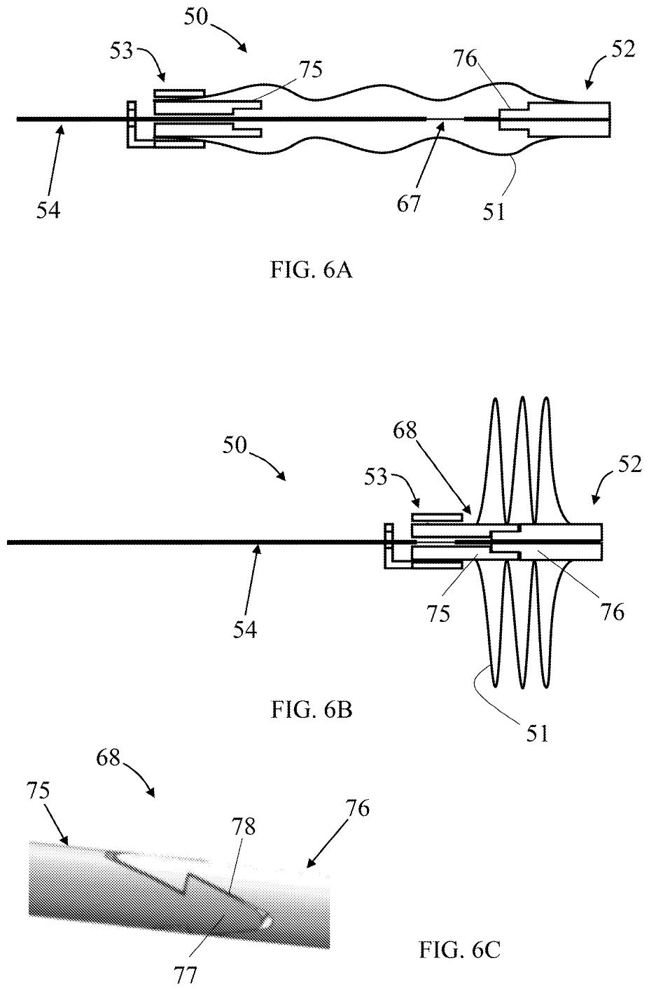

FIGS. 6A-6C schematically illustrate vascular occluder 50 with another different configuration of locking feature 68. Other functional and structural features may be similar or identical to as described with respect to FIGS. 3 and 4. In this exemplary embodiment, locking feature 68 is configured with a proximal locking portion 75 connected to and/or extending from occluder body proximal end 53 and includes a front (distal) surface configured to engage by way of interlocking, bonding or clinging with a rear (proximal) surface of a distal locking portion 76 connected to and/or extending from occluder body distal end 52, so as to lock occluder body 51 in the chosen deployed configuration following compression thereof. In some embodiments, proximal locking portion 75 is configured to interlock with distal locking portion 76 using shaped snapping portions 77 that are shaped and sized to snap-lock into matching recesses 78 of distal locking portion 76, as shown in FIG. 6C for example. In some embodiments, weakened portion 67 is located within or adjacent to proximal locking portion 75 and/or distal locking portion 76 when occluder body 51 is in the chosen deployed configuration.

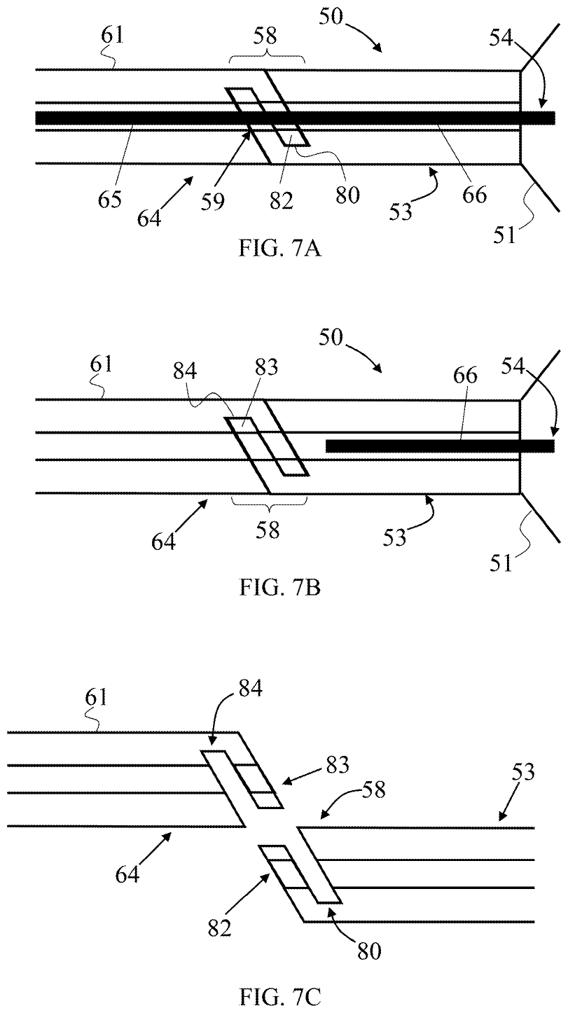

FIGS. 7A-7C schematically illustrate in more details the exemplary connection means used for coupling pusher head 64 with coupling portion 58, and exemplary scenarios in an exemplary sequence for unlocking and releasing coupling portion 58 from pusher head 64. As shown in FIG. 7A, coupling portion 58 includes a coupling portion cavity 80 opened laterally at a side of coupling portion 58. Coupling portion cavity 80 is configured to receive a laterally projecting portion 82 of pusher head 64 configured to fit therein. Laterally projection portion 82 may be sized shaped to match substantially or exactly the size and shape formed by coupling portion cavity 80 therefore allow minimal to no movement therebetween when the two are connected, or alternatively may be smaller in size and allow sufficient room thereby to allow easier connection and/or disconnection therebetween. As shown, both coupling portion cavity 80 and laterally projection portion 82 includes parallel and/or coinciding lumens that together form passage 59 through which tension member 54 extends and tethers laterally projecting portion 82 to coupling portion 80. In some embodiments, connection between vascular occluder 50 and pusher 61 may also be facilitated or strengthen with coupling portion also comprising a lateral projection portion 83 besides its cavity 80, and pusher head also comprising a cavity 84 in which lateral projection 83 can nest of fit. FIG. 7B shows an instance following breaking or dividing of tension member 54, leaving only tension member distal segment 66 connected to occluder body distal end 52 after proximal segment 65 is withdrawn and optionally removed from passage 59, thereby allowing detachment or disengagement of pusher head 64 from coupling portion 58. FIG. 7C schematically illustrates a possible instant were pusher 61 is detached from vascular occluder 50 with pusher head 64 disengaging from coupling portion 58. In some embodiments, laterally projecting portion 82 and/or lateral projection 83 is inclined and angled relative to longitudinal axis of vascular occluder 50, optionally in an angle greater than 25° for example, in order to ease or facilitate detachment of pusher 61 from vascular occluder 50 when pulled along the longitudinal axis.

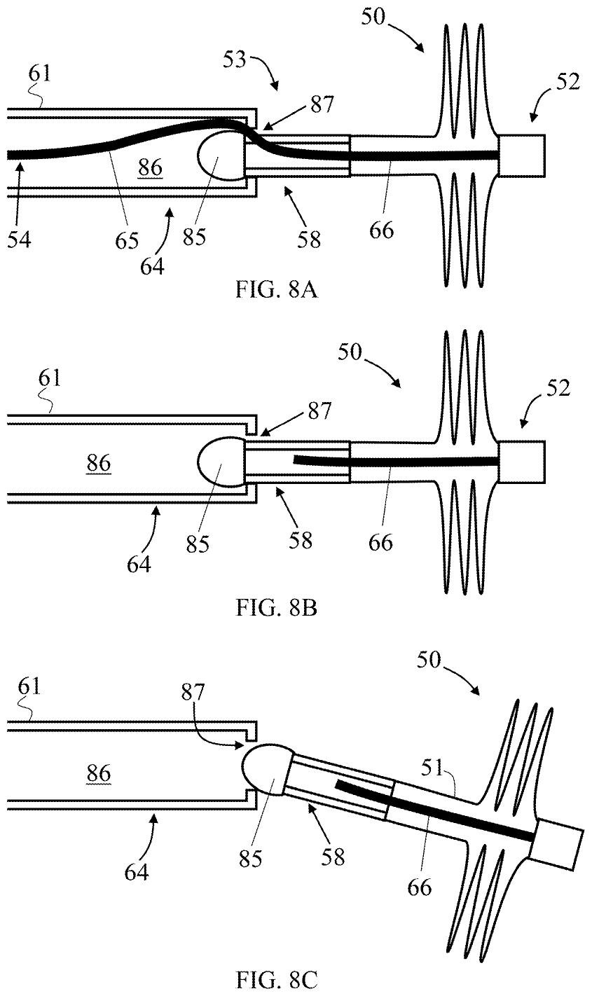

FIGS. 8A-8C schematically illustrate different exemplary connection means used for coupling pusher head 64 with coupling portion 58, and exemplary scenarios in an exemplary sequence for unlocking and releasing coupling portion 58 from pusher head 64. As shown in FIG. 8A, coupling portion 58 includes a widening 85 which may be rounded. Pusher head 64 has a lumen 86 opened via a pusher opening 87 located optionally in a front surface thereof which is facing vascular occluder 50. Vascular occluder 50 is configured such that coupling portion 58 and tension member 54 extend into pusher head lumen 86 via pusher head opening 87, wherein tension member 54 extends in juxtaposition to widening 85 in pusher head lumen 86 adjacent to pusher head opening 87. Widening 85 is sized to pass through the pusher head opening 87 however thickness of tension member 54 adjacent to pusher head opening 87, even when compressed with widening 85, is greater than the maximal gap formable between widening 85 and boundary of pusher head opening 87. Therefore, tension member 54 prevents passing of widening 85 through pusher head opening 87 and therefore the possibility that coupling portion 58 can be disengaged and disconnected from pusher head 64, which otherwise can be attained in absence of tension member 54 passing through pusher head opening 87. FIG. 8B shows an instance following breaking or dividing of tension member 54, leaving only tension member distal segment 66 connected to occluder body distal end 52 after proximal segment 65 is withdrawn and optionally removed from passage 59, thereby allowing detachment or disengagement of pusher head 64 from coupling portion 58. FIG. 7C schematically illustrates a possible instant were pusher 61 is detached from vascular occluder 50 with pusher head 64 disengaging from coupling portion 58.

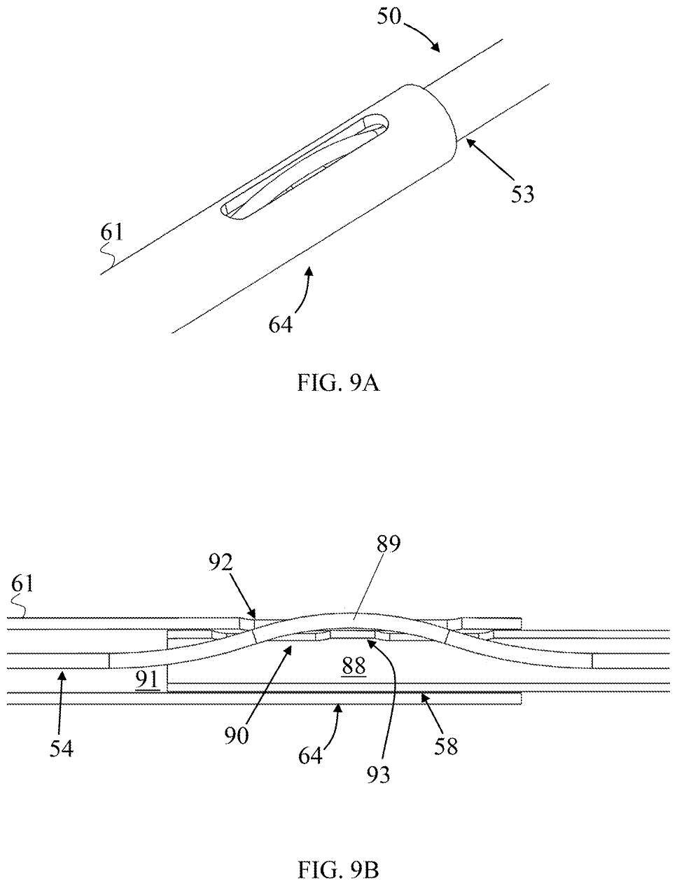

FIGS. 9A-9B schematically illustrate different exemplary connection means used for coupling pusher head 64 with coupling portion 58. In this embodiment, coupling portion 58 is extending along inside (or outside) pusher head 64, optionally concentrically thereto. Coupling portion 58 includes a lumen 88 opened to an at least one side opening 90 on a lateral surface of coupling portion 58. Likewise, pusher head 64 includes a lumen 91 opened to an at least one side opening 92 on a lateral surface thereof. As shown, tension member 54 passes through coupling portion lumen 88 extending along coupling portion 58 and protrudes laterally with a curved portion 89 thereof via coupling portion side opening 90 to an opening area surrounded by pusher head side opening 92 located immediately above coupling portion side opening 90, thereby resisting or preventing longitudinal motion of coupling portion 58 relative to pusher head 64. Coupling portion 58 may include a tab 93 adjacent to the at least one side opening 90, or in between two side openings 90, that is configured to prevent curved portion 89 of tension member 54 from straightening in and aligning with coupling portion lumen 88. When tension member 54 is broken or divided, optionally distally to coupling portion side opening 90 and/or pusher head side opening 92, coupling portion 58 can be disengaged and disconnected from pusher head 64.

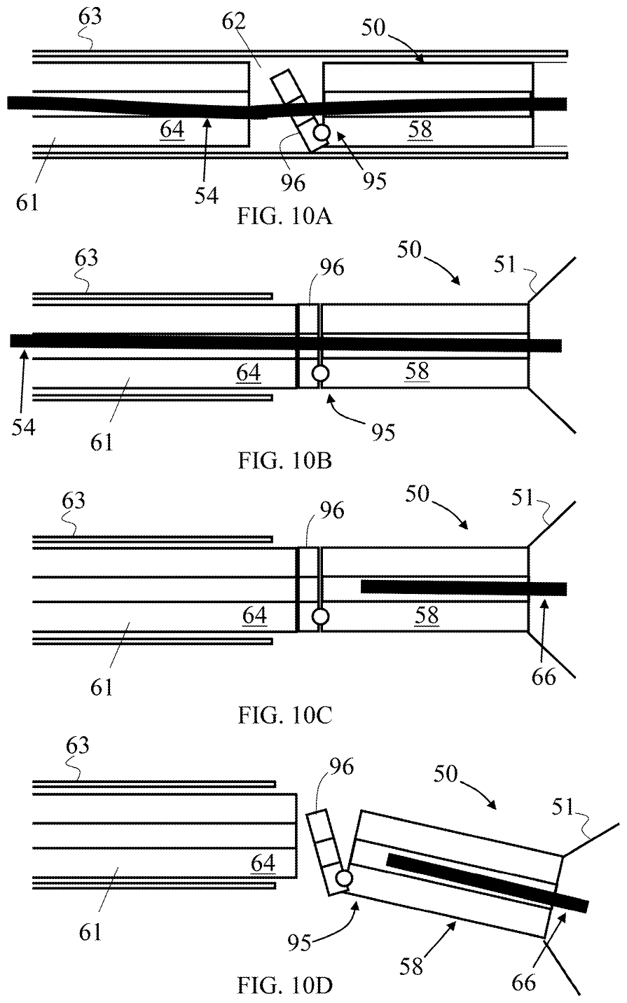

In some embodiments, vascular occluder 50 employs tension member 54 also as tethering means not necessarily for preventing release from connection between coupling portion 58 and pusher head 54, but alternatively or additionally to prevent unintentional delivery, compression and/or release of occluder body 51. Optionally, vascular occluder 50 is configured for facilitating selective retraction thereof by pulling tension member 54 optionally without use of other means such as a pusher. FIGS. 10A-10D schematically illustrate exemplary scenarios in an exemplary sequence for releasing coupling portion 58 which includes an actuator 95 in this embodiment. Actuator 95 is selectively actuatable from a normally opened configuration (shown in FIG. 10A and FIG. 10D) to a closed configuration (shown in FIG. 10B and FIG. 10C). When in the closed configuration, actuator 95 is configured to facilitate unhindered axial motion of the tension member therethrough. When in the normally opened configuration, actuator 95 is configured to engage tension member 54 and to lock it to coupling portion 58 such that axial motion of tension member 54 therethrough is prevented. Actuator 95 includes a lever 96 configured to shift actuator 95 from the normally opened configuration to the closed configuration when pressed to move from a less stressed position to a more stressed position, such as with pusher head 64, as shown in FIG. 10B for example. Actuator 95 is actuatable from the normally opened configuration to the closed configuration when pressed distally under a pushing force sufficient to push occluder body 51 into the bodily lumen portion from catheter 63.

FIG. 10A shows vascular occluder 50 during delivery through lumen 62 of catheter 63 at an instance it is not engaging or in contact with pusher 61, such as during possible retraction vascular occluder 50 by pulling of tension member 54. When not engaged with pusher 61, actuator 95 is in the normally opened configuration with lever 96 engaging with a portion of tension member 54 and locks it to coupling portion 58 such that vascular occluder 50 cannot be moved axially within lumen 62 along and relative to tension member 54. FIG. 10B shows vascular occluder 50 completely uncovered from within catheter 63 with pusher head 64 engaging with actuator 95 and shifting it to its closed configuration by forcing lever 96 to unlock tension member 54 to coupling portion 58. This allows pushing vascular occluder 50 along and relative to tension member 54 by pusher 61 as shown. FIG. 10C shows vascular occluder 50 following breaking or dividing of tension member 54 distally to actuator 95, leaving only tension member distal segment 66 connected to occluder body 51 after proximal segment 65 is withdrawn and optionally removed from passage 59, thereby allowing detachment or disengagement of vascular occluder 50 by the operator, as shown schematically in FIG. 10D for example.

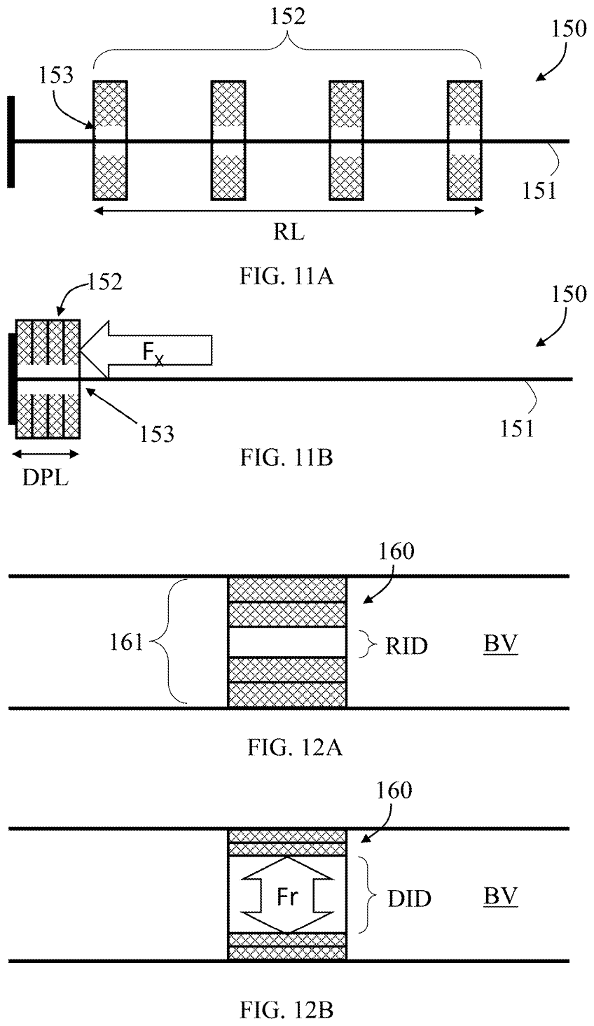

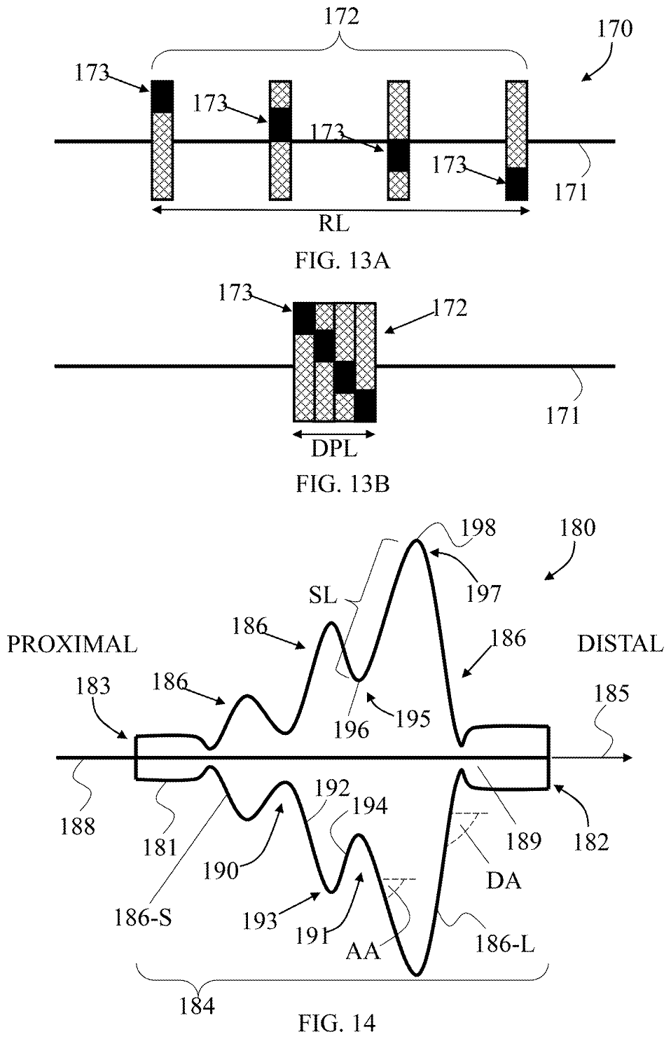

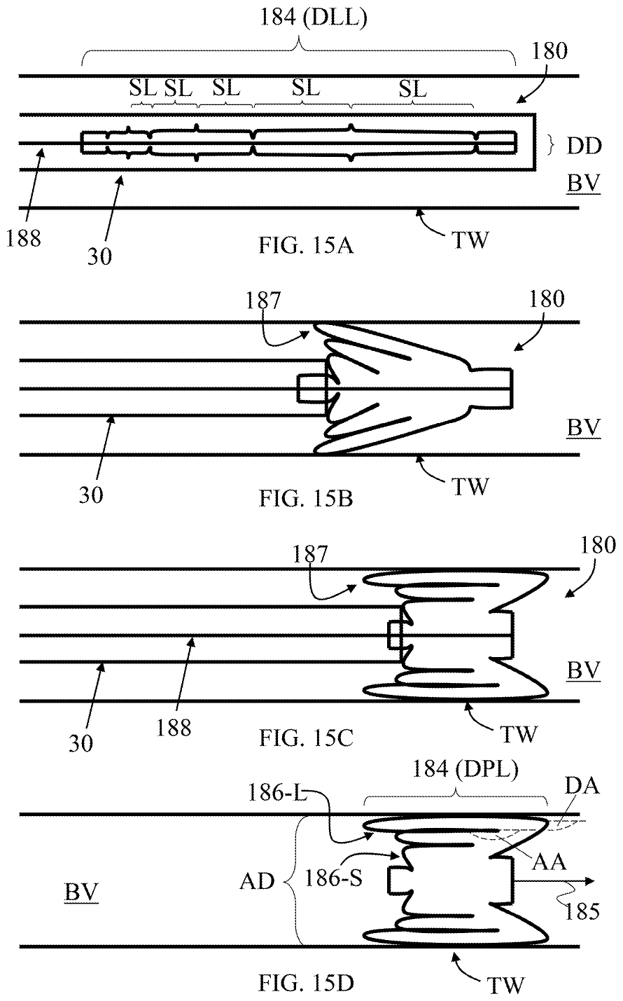

The following FIGS. 11, 12 and 13 depicts three conceptual features, one or more of which may be incorporated in exemplary embodiments described herein. The first conceptual feature illustrated in FIGS. 11A-11B is aimed for selectively affecting reduced permeability of an exemplary vascular occluder 150 when portions thereof are compressed axially against each other (e.g., parallel to longitudinal axis of vascular occluder 150). The second conceptual feature illustrated in FIGS. 12A-12B is aimed for selectively affecting increase in radial strength and/or particularly in resistance to radial inward compression of an exemplary vascular occluder 160 when portions thereof are compressed radially outwardly against each other (e.g., radially to longitudinal axis of vascular occluder 160). The third conceptual feature illustrated in FIGS. 13A-13B is aimed for selectively affecting substantial impermeability of an exemplary vascular occluder 170 when portions thereof are approximated and/or placed in contact each other. Vascular occluder 180 shown in FIGS. 14-15 is provided as an exemplary device designed for encompassing all three conceptual features for use in occluding bodily lumens such as blood vessels.

FIGS. 11A-11B schematically illustrate vascular occluder 150 which may be an exemplary implementation or variation of vascular access port 10 and/or 50. vascular occluder 150 comprising of a tension member 151 and a plurality of flow obstruction portions 152 provided sequentially along a portion of tension member 151 and extending at least partially transversely thereto. Each one of the flow obstruction portions 152 is partially permeable to blood flowing therethrough and is sized and configured to cover most or all cross section of the bodily lumen portion it is prescribed to treat. Optionally, each one of the flow obstruction portions 152 includes a mesh pattern and/or a braided or interwoven structure with mesh openings sized to allow blood flow therethrough, yet optionally to cause some degree of obstruction thereto. Tension member 151 passes through a respective lumen 153 of each one of the flow obstruction portions 152 such that one or more of the plurality of flow obstruction portions 152 is movable axially over the tension member 151 relative to remainder of the flow obstruction portions.

Flow obstruction portions 152 are selectively compressible against each other axially (e.g., in a direction parallel to the tension member and/or the route defined by the bodily lumen treated) by a longitudinal (axial) compression force Fx, from a more elastically relaxed length RL (FIG. 11A) to a fixable deployed length DPL (FIG. 11B), such that radially inner portions thereof are pressed tight against each other. Deployed length DPL can be fixated or locked by self-locking mechanism or structure and/or by using locking-specific means.

Vascular occluder 150 is configured less permeable to blood flowing therethrough in the bodily lumen when the plurality of flow obstruction portions 152 is in the deployed length DPL than when the plurality of flow obstruction portions 152 is in the more elastically relaxed length RL. In some embodiments, reduction in permeability results from the particular fluid permeable structure of each one of the obstruction portions 152 that derives a specific (e.g., predetermined fluid flow pattern or regime therethrough, while approximating and/or contacting the plurality of flow obstruction portions 152 axially into a smaller volume further affects the flow regime by increasing the density of obstacles to axial flow therethrough. In similar or other embodiments, each one of the flow obstruction portions 152 is forced to compress to a thinner form while causing structural deformation that reduces its own permeability to fluid flowing therethrough.

The plurality of flow obstruction portions 152 may be provided as separate (distinct) members as shown, or they may be portions of a single structure, as will demonstrated in a particular exemplary embodiment with respect to vascular occluder 180. In some embodiments, vascular occluder 150 includes an elongated occluder body comprising an occluding section wherein the plurality of flow obstruction portions 152 forms the occluding section or part thereof.

FIGS. 12A-12B schematically illustrate vascular occluder 160 which may be an exemplary implementation or variation of vascular access port 10, 50 and/or 150. Vascular occluder 160 comprising a plurality of anchoring portions 161 provided concentrically one over the other at a target portion of a blood vessel BV. In some embodiments, the plurality of anchoring portions 161 form together a structure configured to obstruct a normal flow of blood therethrough in the blood vessel BV, and each one of the anchoring portions 161 is optionally partially permeable to blood flowing therethrough. Optionally, each one of anchoring portions 161 includes a mesh pattern and/or a braided or interwoven structure with mesh openings sized to allow blood flow therethrough, yet optionally to cause some degree of obstruction thereto.

Vascular occluder 160 is configured to self-anchor to wall of blood vessel BV by expanding to a more elastically relaxed configuration such that it becomes in contact with periphery (radially outer portion) thereof with the blood vessel wall and optionally forces the blood vessel wall to expand locally. In some embodiments, the resistance to radial inward compression in this more elastically relaxed configuration is insufficient for continuous or permanent anchoring and for preventing migration, for example when local diameter of the blood vessel wall portion is in constant change such as due to wall muscle activity and changes between systolic and diastolic pressures.

In order to overcome such potential issues, vascular occluder 160 is configured to selectively increase its resistance to radial inward compression by the blood vessel wall. As such, anchoring portions 161 are selectively compressible radially outwardly against each other and against wall of blood vessel BV (e.g., in a direction orthogonal to the route defined by the bodily lumen treated, in this case blood vessel BV), by selectively applying a radial force or pressure Fr thereto, from a more elastically relaxed inner diameter RID (FIG. 12A) to a fixable deployed inner diameter DID (FIG. 12B), such that radially inner portions 162 thereof are pressed tight against each other. Deployed inner diameter DID can be fixated or locked by self-locking mechanism or structure and/or by using locking-specific means. When locked in the deployed inner diameter DID, the plurality of anchoring portions 161 provides substantially greater resistance to radial inward compression than when the plurality of anchoring portions 161 is in the more elastically relaxed inner diameter RIL.

In some embodiments, increase in radial strength and/or resistance to radial compression results from a continuous application of outward force (or pressure) that forces inner anchoring portions against the outer anchoring portion, when vascular occluder 160 is locked in the deployed configuration. In similar or other embodiments, each one of the anchoring portions 161 is forced to compress elastically to a thinner form while causing structural deformation that increases the overall springback of the plurality of anchoring portions 161 such that greater forces are then required to compress the vascular occluder to a smaller outer diameter.

The plurality of anchoring portions 161 may be provided as separate (distinct) members, or they may be portions of a single structure, as will demonstrated in a particular exemplary embodiment with respect to vascular occluder 180. In some embodiments, vascular occluder 160 includes an elongated occluder body comprising an occluding section wherein the plurality of anchoring portions 152 forms the occluding section or part thereof.

FIGS. 13A-13B schematically illustrate vascular occluder 170 which may be an exemplary implementation or variation of vascular access port 10, 50, 150 and/or 160. Vascular occluder 170 comprising of a tension member 171 and a plurality of flow obstruction portions 172 provided sequentially along a portion of tension member 171 and extending at least partially transversely thereto. One or more of the plurality of flow obstruction portions 172 is movable axially over the tension member 171 relative to remainder of the flow obstruction portions. Each one of the flow obstruction portions 172 is partially permeable to blood flowing therethrough and is sized and configured to cover most or all cross section of the bodily lumen portion it is prescribed to treat. Furthermore, each one of the flow obstruction portions 172 includes at least one region 173 that is less permeable to blood flow therethrough than remainder of the particular flow obstruction portion, such that blood will normally tend to flow around region 173 through remainder of the flow obstruction portion than through region 173. In some embodiments, region 173 is impermeable to blood flow.

Optionally, each one of the flow obstruction portions 172 includes a mesh pattern and/or a braided or interwoven structure with mesh openings sized to allow blood flow therethrough, yet optionally to cause some degree of obstruction thereto. In some such embodiments, less permeable region 173 will have denser pattern and/or thicker yarns for example or will be made impermeable using a localized covering or impregnated material over the mesh pattern. As shown, flow obstructing portions 172 vary in radial location of their respective less permeable region 173, meaning that in some or all flow obstructing portions 172 the respective region 173 will be located in a different radial distance and/or radial direction away from centerline and/or longitudinal axis of vascular occluder 170. This means that a stream pattern of blood caused by passing through one of the flow obstructing portions 172 will change when approaching and/or passing through the next flow obstructing portion due to the tendency to avoid or flow around region 173 of a different radial location.

As such, vascular occluder 170 is configured less permeable to blood flowing therethrough in the bodily lumen when the plurality of flow obstruction portions 172 is in the deployed length DPL (as shown in FIG. 13A) than when the plurality of flow obstruction portions 172 is in the more elastically relaxed length RL (as shown in FIG. 13B). This may happen for example since that when the flow obstructing portions are distant from each other the blood flow can maneuver and adapt its flow pattern in between each consecutive flow obstruction portions 172, while when the flow obstruction portions are in contact with each other in a compressed form the blood has fewer cross sectional areas not covered with any of the less permeable or impermeable regions 173 forcing them either to flow through or be blocked by regions 173.

Additionally or alternatively, regions 173 changes in permeability to blood flow when and/or as a result of the longitudinal (axial) compression of flow obstruction portions 172, collectively forming an obstructing section of vascular occluder 170, into a deployed configuration resulting in deployed length DPL. Change in permeability can be affected for example when structural openings or pores in regions 173 reduces in size and/or when regions 173 become denser, for example due to compression thereof. Such change in permeability may cause reduced flow rate of blood passing through regions 173 or complete blockage thereof, comparing to the flow pattern of the structural and/or functional state of flow obstruction portions 172 in the more elastically relaxed length RL. The plurality of flow obstruction portions 172 may be provided as separate (distinct) members as shown, or they may be portions of a single structure, as will demonstrated in a particular exemplary embodiment with respect to vascular occluder 180. In some embodiments, vascular occluder 170 includes an elongated occluder body comprising an occluding section wherein the plurality of flow obstruction portions 172 forms the occluding section or part thereof.

FIG. 14 schematically illustrates exemplary vascular occluder 180 comprising a plurality of bellows sections varying in diameter, and FIGS. 15A-15D schematically illustrate exemplary scenarios representing steps in an exemplary procedure for implanting vascular occluder 180 in a bodily lumen such as blood vessel BV. Vascular occluder 180 includes an elongated occluder body 181 comprising a distal body end 182, a proximal body end 183 and an occluding section 184 extending along a longitudinal axis 185 between distal body end 182 and proximal body end 183. Vascular occluder 180 may be an exemplary implementation or variation of vascular access port 10, 50, 150, 160 and/or 170, and/or it may include some or all embodiments and features of vascular access port 10, 50, 150, 160 and/or 170, and/or be identical or similar in function, structure and/or modality to one or more of vascular access ports 10, 50, 150, 160, and 170.

Vascular occluder 180 is shown in FIG. 14 in an elastically relaxed configuration wherein occluding section 184 forms a tubular bellows-like shaped structure comprising a plurality of bellows sections 186. From this state, occluding section 184 is elastically stretchable longitudinally into a delivery configuration (shown in FIG. 15A, for example) having a delivery length DLL and a delivery diameter DD, and elastically compressible longitudinally into a deployed configuration (shown in FIG. 15D, for example) having a deployed length DPL smaller than the delivery length DLL and a deployed diameter AD greater than delivery diameter DD. Deployed length is optionally about 50% or less than delivery length DLL. Delivery length DLL is optionally greater than about 10 mm, and the deployed length DPL is optionally smaller than about 6 mm, and delivery diameter DD is optionally smaller than about 2 mm with deployed diameter AD is optionally greater than about 5 mm.

Occluder body 181 (including occluding section 184) is formed of a braided or interwoven sleeve. The sleeve may be formed of metal wires such as Nickel-Titanium alloy wires, optionally about 20 to 200 wires, optionally of about 25 to about 150 micrometer in diameter each. The braid pattern may include a portion of wires of a radiopaque metal, optionally containing Platinum or Palladium, for improved visibility under fluoroscopic imaging. The braiding may be executed either manually or by using a braiding machine. Sleeve outer diameter may be constant in diameter and its size may vary and be approximated to average internal diameter of the target vessel. Following sleeve braiding, it can then undergo a shape setting treatment, bringing it to its resting (relaxed) state configuration. To accomplish that, the sleeve can be installed on a bellows-shaped mandrel of chosen shape and dimensions, forcing it to the desired shape, followed by annealing phase (which may include heating at a temperature of 500-650° c. for a duration of 2-15 minutes, for example, followed by a rapid cooling in water or oil). The braided sleeve may then undergo complimentary metallurgical treatments to improve its corrosion and fatigue resistance.

Vascular occluder 180 is configured such that, when occluding section 184 is in the deployed configuration, radially inner portions of the plurality of bellows sections 186 and/or of distal and proximal body ends 182 and 183 are fixedly pressed tight against each other parallel to the longitudinal axis 185. Vascular occluder 180 may be configured such that, when the occluding section 184 is in the deployed configuration, the radially inner portions are deformed into a condensed form with no gap therebetween. Furthermore, when occluding section 184 is in the deployed configuration, radially outer portions of the plurality of bellows sections 186 are optionally configured to be spaced radially outwardly from the radially inner portions, are allowed to shift relative to each other and/or to be arranged with gaps therebetween.

Occluding section 184 is configured to lock in the deployed configuration after having been compressed longitudinally thereto. The occluding section 184 is configured to lock (e.g., self-lock) in the deployed configuration when at least one of bellows sections 186 is held bent towards longitudinal axis 185, optionally particularly if bent towards proximal body end 183. When locked this way, at least one of the bellows sections 186 is held deformed such that a radially outer portion 187 thereof is bent towards longitudinal axis 185. In such or other embodiments, a length securing member (which may optionally include use of a tension member) is provided with or as part of vascular occluder 180, which is configured to lock occluding section 184, selectively or automatically, in the deployed configuration by resisting lengthwise changes from the deployed length DPL.

Vascular occluder 180 also includes a tension member 188 extending along occluding section 184 and connected with a distal end thereof to a distal end of occluding section 184 (in this example, it adjoins with distal body end 182). Tension member 188 is configured to facilitate selective longitudinal compression of occluding section 184 such as by applying tension (e.g., by pulling with sufficient force in a proximal direction) to tension member 188 when a proximal end of the occluding section (in this example, it adjoins with proximal body end 183) is pushed in a distal direction. The tension member 188 is optionally connectable to proximal end of occluding section 184 (e.g., to proximal body end 183) when it is in the deployed configuration, for preventing elongation of occluding section 184 from the deployed length DPL, and as such can serve as a length securing member or part in a length securing mechanism.

In some embodiments, occluding section 184 encloses a lumen 189 extending along the longitudinal axis 185 with tension member 188 extending therethrough. In some embodiments, tension member 188 is configured to separate or cut to a predetermined length, optionally when pulled by a force greater than a predetermined tension force. Tension member 188 may also be releasably fixated with the distal end thereof to the distal end of occluding section 184, and configured to disconnect therefrom, optionally when pulled by a force greater than a predetermined tension force.

Occluding section 184 is configured to reduce blood flow rate passing therethrough along the longitudinal axis 185 when compressing longitudinally to the deployed configuration shown in FIGS. 15C and 15D from a less elastically stressed longitudinally compressed state shown in FIG. 15B. Occluding section 184 is configured to increase in maximal outer diameter when compressing longitudinally to the deployed configuration from the less elastically stressed longitudinally compressed state. Furthermore, occluding section 184 is configured to increase in elastic resistance to radial compression, when compressing longitudinally to the deployed configuration, from the less elastically stressed longitudinally compressed state.

At least one of the plurality of bellows sections 186 extends between respective first 190 and second 191 inward creases, and comprises a respective frustum-shaped ascending portion 192 extending from the respective first inward crease 190 to a respective outward crease 193, and a respective frustum-shaped descending portion 194 extending from the respective outward crease 193 to the respective second inward crease 191. At least one of the respective frustum-shaped ascending portion 192 and frustum-shaped descending portion 194 is configured to reduce blood flow permeability therethrough along the longitudinal axis 185, when occluding section 184 changes to the deployed configuration.

In each pair of adjacent bellows sections 186 comprising of respective first and second bellows sections, the respective second inward crease 191 of the respective first bellows section and the respective first inward crease 190 of the second bellows section are adjoined circumferentially at a mutual curved or bent circumferential inner edge 195 having an inward apex 196 projecting radially-inwardly towards the longitudinal axis 185. The respective outward crease 193 includes a curved or bent circumferential outer edge 197 having an outward apex 198 projecting radially-outwardly from the longitudinal axis 185.

In some embodiments and as shown, at least some of the plurality of bellows sections 186 vary in enclosed largest diameter, and in at least one of the plurality of bellows sections 186 a smallest diameter enclosed by the respective second inward crease 191 is greater than a smallest diameter enclosed by the respective first inward crease 190 and smaller than a largest diameter enclosed by the outward crease 193. In some such embodiments, in each three sequentially positioned bellows sections of the plurality of bellows sections 186, comprising a respective second bellows section longitudinally positioned between a respective first bellows section and a respective third bellows section, the respective largest diameter enclosed by the respective second bellows section is equal to or greater than the respective largest diameter enclosed by the respective first bellows section and is equal to or smaller than the respective largest diameter enclosed by the respective third bellows section.

At least one of the plurality of bellows sections 186 is less permeable adjacent to respective first inward crease 190, second inward crease 191 and/or outward crease 193 than to remainder of the respective frustum-shaped ascending portion 192 and/or frustum-shaped descending portion 194, to blood flow passing therethrough along the longitudinal axis. As such, the regions at and adjacent to the creases are considered less permeable (or optionally impermeable) regions similar to regions 173 described above. In some embodiments and as shown, the different creases in each bellows section 186 and across most or all bellows sections 186 vary in radial location (e.g., distance) from longitudinal axis 185, therefore when occluding section 184 is longitudinally compressed in the deployed configuration, these less permeable regions cause immediate drop in permeability.

In some embodiments and as shown, the plurality of bellows sections 186 includes at least one larger bellows section 186-L having the respective outward crease 193 thereof enclosing a largest diameter greater than diameter of the treated bodily lumen portion, and at least one smaller bellows section 186-S having the respective outward crease thereof 193 enclosing a largest diameter equal to or smaller than diameter of the treated bodily lumen portion. As such, the radially outer portions of at least larger bellows section 186-L and smaller bellows section 186-5 are considered anchoring portions similar to anchoring portions 161 described above. As such, vascular occluder 180 is configured such that when occluding section 183 is in the deployed configuration, the at least one smaller bellows section 186-5 is located inside larger bellows section 186-L and presses radially outwardly against the larger bellows section 186-L, thereby increasing anchoring pressure towards the blood vessel wall and/or increases resistance to radial inward compression.

In some embodiments and as shown, in at least one of the plurality of bellows sections 186 the respective frustrum-shaped ascending portion 192 is different in slant length SL than the respective frustum-shaped descending portion. Additionally or alternatively, at least some of the plurality of bellows sections 186 may vary in slant length SL of the respective frustum-shaped ascending portion 192 and/or frustum-shaped descending portion 194 thereof. Similarly, at least some of the plurality of bellows sections 186 vary in cumulative (total) slant length of the respective frustum-shaped ascending portion 192 and frustum-shaped descending portion 194 thereof.

The respective frustum-shaped ascending portion 192 forms an ascending angle AA with longitudinal axis 185 and the respective frustum-shaped descending portion 194 forms a descending angle DA, with and in the direction of longitudinal axis 185. In some embodiments and as shown, when the occluding section 184 is in the elastically relaxed configuration, ascending angle AA is smaller than 90° and descending angle DA is greater than 90°. When occluding section 184 is in the deployed configuration, each one of ascending angle AA and descending angle DA is either greater or smaller than 90° (in this example and as shown, both angles are greater than 90°).

A method of occluding a blood vessel BV may include a sequence or a number of steps that include one or more of the scenarios shown in FIGS. 15A-15D. Vascular occluder 180 is inserted into blood vessel BV proximately to a target wall portion TW of blood vessel BV when the occluding section 184 is held restrained in the delivery configuration (FIG. 15A). Delivery is performed using a delivery catheter such as catheter 30, and once in place the vascular occluder 180 is ejected from lumen of the delivery catheter (such as by using pusher 31), for allowing occluding section 184 to be released in blood vessel BV by shortening and expanding laterally into a less elastically stressed configuration (as shown in FIG. 15B). In some embodiments, occluding section 184 can be selectively maintained (e.g., manually or with a locking mechanism, for example) in the delivery configuration and in delivery length DLL also after partially or fully uncovering it from catheter 30, until the operator chooses to release and/or compress it to a smaller length. Alternatively, vascular occluder 180 can be operated (automatically or selectively) such that upon uncovering from catheter 30, occluding section 184 is immediately released, gradually during uncovering process or fully upon complete uncovering, into a less elastically stressed configuration, such as the one illustrated in FIG. 15B, for example.

As shown in FIG. 15C, occluding section 184 can then be compressed longitudinally by applying opposite forces to distal and proximal ends of occluding section 184 (optionally by pushing catheter 30 and/or pulling tension member 188) into the deployed configuration, thereby increasing longitudinal elastic stress of occluding section 184 relative to the less elastically stressed configuration. This forceful longitudinal compression, and in view of the diameter varying design of the bellows sections 186 described above, causes the at least one smaller bellows section 186-S to press radially outwardly against the larger bellows section 186-L thereby increasing grip and resistance to inward compression by the target wall portion TW.

The compression may also include or be accomplished by pressing tight radially inner portions of the plurality of bellows sections 186 and/or of the distal and proximal body ends 182 and 183 against each other parallelly to the longitudinal axis 185, which optionally causes reduced permeability of the occluding section 184 to blood flowing longitudinally therethrough. FIG. 15D shows vascular occluder 180 with occluding section locked in the deployed configuration, either due to its self-locking design once compressed in the deployed configuration (as shown), or when locked using a length securing member (as shown in FIG. 1C, for example).

Releasing or compressing longitudinally occluding section 184 as described forces bending of at least one of the bellows sections 186 towards the longitudinal axis 185, and then the compressing longitudinally or locking generates continuous internal stresses configured to hold the at least one of the bellows sections 186 bent towards the longitudinal axis 185, thereby locking occluding section 184 in the deployed configuration.

In case the locking includes use of tension member 188, a portion thereof can be connected to a proximal end of occluding section 184 when in the deployed configuration, and the remainder portion of tension member 188 proximally to occluding portion 184 can be disjoined or cut, optionally to a predetermined length, such as by applying tension thereto above a chosen tension force. Alternatively, in case tension member 188 is not used for holding occluding section 184 locked but only for applying compression thereto, the step of locking occluding section 184 can be followed by releasing or disconnecting tension member 188 from the distal end of occluding section 184, such as by cutting and/or by applying tension thereto above a chosen tension force.

In some embodiments, the occluding section of the vascular occluder is less permeable to blood flowing therethrough when in the deployed configuration relative to when in the elastically relaxed configuration. The flow obstruction portions may be at least partially permeable to blood flowing therethrough, and in some such embodiments the occluding section also includes an impermeable portion configured to block blood from flowing through the occluding section along the longitudinal axis, when in the deployed configuration. In some embodiments, the impermeable portion is not completely impermeable but characterized with substantially low permeability and/or substantially more thrombogenic than other portions of the occluding sections.

The impermeable portion may include, or be configured as, a coating of inner or outer portions of the occluding sections and/or a radially expandable member housed in the occluding section between the flow obstruction portions. Alternatively, the impermeable portion may include, or be configured as, an at least one axially extendable-compressible member extending through the occluding section and configured to allow unhindered stretching of the occluding section to the delivery configuration, and to compress into a flattened or condensed form spanning over part or substantially all axial cross-sectional area of the occluding section when in the deployed configuration thereby occluding the occluder body. In some such embodiments, the impermeable portion may be configured to allow blood flow through the occluding section when in the elastically relaxed configuration.

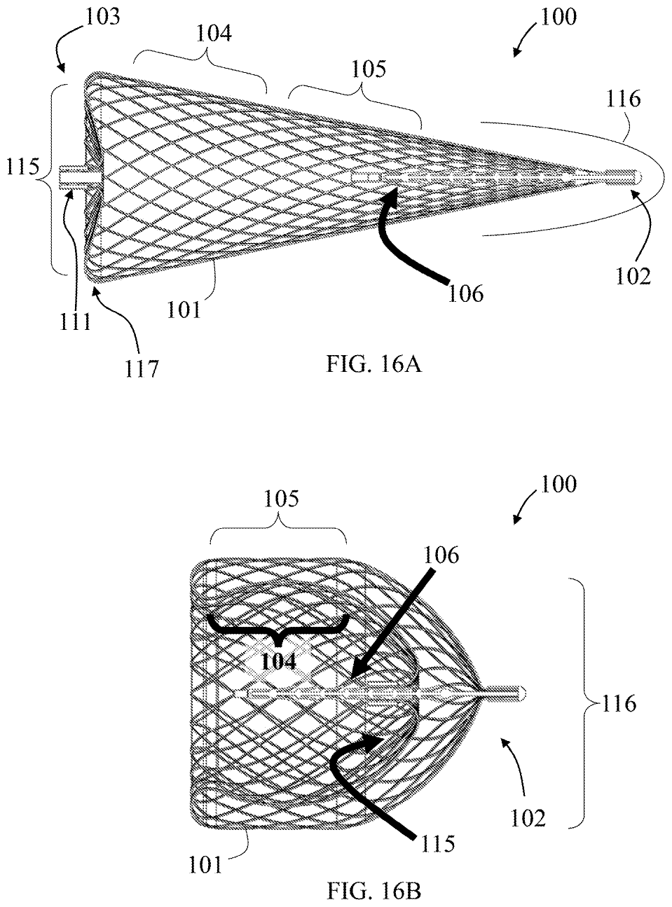

FIGS. 16A-16B illustrate cross-sectional side views of an exemplary vascular occluder 100 in a preliminary deployed configuration (FIG. 16A) and in a compressed deployed configuration (FIG. 16B). Vascular occluder 100 may be similar or identical to at least one of vascular occluders 10, 50 and 180, at least in part, optionally as an exemplary variation thereof, and/or may include one or more similar or identical structural and/or functional feature described above. Vascular occluder 100 includes an occluder body 101 extending longitudinally between an occluder body distal end 102 and an occluder body proximal end 103. Occluder body 101 is at least partially meshed, woven and/or braided, and is at least partially impermeable and/or is covered or impregnated with an impermeable or hydrophilic substance or material. In some embodiments, occluder body 101 is formed of a plurality of wires braided together to form a tubular shape (e.g., conic, frustum or cylindric-like shape) optionally more than 10 wires, optionally more than 30 wires (e.g., 32 or 48 wires in some specific exemplary embodiments). The wires may be formed of an elastic metal or metal alloy (e.g., Ni—Ti alloy), optionally each wire has an average diameter taken between 10 and 500 microns (μm), optionally between 50 and 200 microns (e.g., 67, 75, or 100 microns in some specific exemplary embodiments).

In some embodiments, occluder body 101 is configured to substantially reduce blood flow passing therethrough but optionally not to fully block it (e.g., by fully occluding the blood vessel) for allowing hemodynamic process of blood clots to form on occluder body 101. In some embodiments, occluder body 101 is formed of a material and/or a weaving (e.g., braiding) pattern configured for resisting blood flow for reducing rate thereof, optionally by way of possessing total contact surface area with the blood flowing therethrough being substantially greater than the cross-sectional area of the blood vessel it occludes. Occluder body 101 includes a proximal occluding section 115 at or adjacent to distal end 102 (when in the preliminary deployed configuration) and a distal occluding section 116 at or adjacent to occluder body proximal end 103. At least one of proximal occluding section 115 and distal occluding section 116 is configured to cover and/or at least partially occlude most or all cross-sectional area of a passage, when occluder body 101 is in the compressed deployed configuration. Proximal occluding section 115 and distal occluding section 116 include a meshed, braided and/or porous pattern and/or material across most or all area thereof, that is configured to reduce flow velocity and/or to prevent localized increase of flow velocity of blood passing therethrough, for example when the occluder body is in the compressed deployed configuration. In some embodiments, when proximal occluding section 115 and distal occluding section 116 are brought closer together and/or adjacent with each other, the overall permeability to blood flow along occluder body 101 is reduced, similarly to as described with respect to vascular occluder 180. In some embodiments, occluder body 101, and/or proximal occluding section 115 and distal occluding section 116, is formed with woven or braided wires, configured with chosen density, porosity and/or fiber amorphousness, and as such is designed and configured to allow some permeability while preventing local increase of blood flow velocity commonly associated with venturi effect through small holes. Preventing localized high velocity blood flow can increase likelihood of proper blood clotting around and/or within occluder body 101, for gradually occluding the blood vessel until full occlusion (blockage).

Occluder body 101 is elastically compressible longitudinally from the preliminary deployed configuration to the compressed deployed configuration. When in the preliminary deployed configuration, occluder body 101 is configured with a preliminary deployed length and a preliminary deployed diameter (e.g., average diameter), and when in the compressed deployed configuration, occluder body 101 is configured with a compressed deployed length smaller than the preliminary deployed length and a compressed deployed diameter (e.g., maximal or average diameter) equal to or greater than the preliminary deployed diameter. Furthermore, occluder body 101 is elastically stretchable longitudinally into a delivery configuration having a delivery length substantially greater than preliminary deployed length, and a delivery diameter substantially smaller than the preliminary deployed diameter. In some embodiments, the delivery length of occluder body 101 is at least about 5 mm, optionally at least about 10 mm, optionally at least about 20 mm (optionally about 20 mm, about 30 mm, about 40 mm, or about 60 mm, in some specific exemplary embodiments). In some embodiments, the preliminary deployed length and/or the compressed deployed length is smaller than the delivery length, for example its can be between about 20% and about 80%, or optionally between about 40% and about 60%, of the delivery length. In some embodiments, the compressed deployed length is smaller than the preliminary deployed length by at least 15% optionally by at least 25%, or optionally by at least 40%. In some embodiments, the delivery diameter of occluder body 101 is about 2 mm or less, optionally about 1.5 mm or less (optionally about 1 mm, about 1.2 mm, about 1.4 mm, or about 1.8 mm, in some specific exemplary embodiments).

In some embodiments, the preliminary deployed diameter and/or the compressed deployed diameter is greater than the delivery diameter, and it can be for example at least 3-times, optionally at least 4-times, optionally at least 5-times, optionally at least 6-times, or optionally at least 10-times, the delivery diameter. In some exemplary embodiments, the compressed deployed diameter is equal to or greater than target vessel inner diameter which can be for example between 5 mm and 8 mm, optionally between 7 mm and 10 mm, optionally between 9 mm and 12 mm, or optionally at least 12 mm. In some embodiments, the compressed deployed diameter is substantially equal to the preliminary deployed diameter, and in some other embodiments it is greater than the preliminary deployed diameter such as by at least 5% optionally by at least 10%, optionally by at least 25%, or optionally by at least 40%. In some embodiments, a ratio of maximal outer diameter to length of occluder body 101 is increased by at least 500%, optionally by at least 700%, or optionally by at least 900% between the delivery configuration and the compressed deployed configuration, and/or that a ratio of the delivery diameter to the delivery length is equal to or smaller than about 0.1, or than about 0.7, or than about 0.05, and/or the ratio of the compressed deployed diameter to the compressed deployed length is greater than about 0.3, or than about 0.5, or than about 0.7.

In one exemplary embodiment, for treating a blood vessel portion (e.g., of an artery) sized between about 5 mm and about 8 mm in diameter, chosen vascular occluder 100 can have occluder body 101 with a preliminary deployed diameter of about 5 mm, and be configured for being selectively compressed (e.g., gradually and/or sequentially) by a user to a compressed deployed diameter being greater than the original treated blood vessel portion diameter (before insertion of occluder body 101 thereto) by about 10% or more, optionally up to about 10 mm or up to 140% the original blood vessel portion diameter. In a second exemplary embodiment, for treating a blood vessel portion (e.g., of an artery) sized between about 7 mm and about 10 mm in diameter, chosen vascular occluder 100 can have occluder body 101 with a preliminary deployed diameter of about 7 mm, and be configured for being selectively compressed (e.g., gradually and/or sequentially) by a user to a compressed deployed diameter being greater than the original treated blood vessel portion diameter (before insertion of occluder body 101 thereto) by about 10% or more, optionally up to about 14 mm or up to 140% the original blood vessel portion diameter. In a third exemplary embodiment, for treating a blood vessel portion (e.g., of an artery) sized between about 9 mm and about 12 mm in diameter, chosen vascular occluder 100 can have occluder body 101 with a preliminary deployed diameter of about 9 mm, and be configured for being selectively compressed (e.g., gradually and/or sequentially) by a user to a compressed deployed diameter being greater than the original treated blood vessel portion diameter (before insertion of occluder body 101 thereto) by about 10% or more, optionally up to about 18 mm or up to 140% the original blood vessel portion diameter. In a fourth exemplary embodiment, for treating a blood vessel portion (e.g., of an artery) greater than about 12 mm in diameter, chosen vascular occluder 100 can have occluder body 101 with a preliminary deployed diameter of about 12 mm, and be configured for being selectively compressed (e.g., gradually and/or sequentially) by a user to a compressed deployed diameter being greater than the original treated blood vessel portion diameter (before insertion of occluder body 101 thereto) by about 10% or more, optionally up to about 24 mm or up to 140% the original blood vessel portion diameter.

When in the preliminary deployed configuration, occluder body 101 reduces in diameter, at least in part thereof and/or optionally gradually, in a direction from proximal to distal, optionally forming a cone-like of frustum-like shape pointing distally. When in the compressed deployed configuration, at least part of occluder body 101 is substantially cylindrical and/or a front portion thereof becomes more rounded, relative to its shape in the preliminary deployed configuration, and it optionally forms a dome-like shape pointing distally. Occluder body 101 includes a proximal tubular body segment 104 and a distal tubular body segment 105. When the occluder body is in the preliminary deployed configuration, proximal tubular body segment 104 is greater in average diameter, and/or greater in diameter along most or length thereof, than distal tubular body segment 105. When occluder body 101 is in the preliminary deployed configuration, proximal tubular body segment 104 extends proximally to distal tubular body segment 105 and the two form a single layer extending between occluder body proximal end 103 and occluder body distal end 102.

Occluder body proximal end 103 and proximal occluding section 115 are optionally located proximally to rest of occluder body 101 when in the preliminary deployed configuration, and are optionally configured to be displaced distally more than other portions of occluder body 101 (optionally particularly inner or central portion thereof), when shifting to the compressed deployed configuration, such that they located distally to proximal tubular body segment 104 when occluder body 101 is in the compressed deployed configuration. When occluder body 101 changes from the preliminary deployed configuration to the compressed deployed configuration, proximal tubular body segment 104 is inserted into, and/or inwardly folded into, and/or tucked in, and/or stuffs, distal tubular body segment 105, thereby forcing the distal tubular body segment 105 to expand radially and/or to reshape elastically. When advanced or pushed distally, proximal tubular body segment 104 is gradually inverted and forms an inner layer relative to distal tubular body segment 105 until reaching the compressed deployed configuration. In some embodiments, a portion of occluder body 101 includes a circular crease 117 and/or is configured to facilitate a fold when proximal tubular body segment 104 is forced to advance (e.g., when changing from the preliminary deployed configuration to the compressed deployed configuration) towards distal tubular body segment 105, thereby allowing a predetermined folding shape and pattern to guide the shape change of the proximal tubular body segment 104 when inserting proximal tubular body segment 104 inside of distal tubular body segment 105. Crease 117 may be located between proximal occluding section 115 and distal tubular body segment 105, optionally particularly between proximal occluding section 115 and proximal tubular body segment 104 (as shown) or between proximal tubular body segment 104 and distal tubular body segment 105.

Vascular occluder 100 further includes a tension member 106 configured to tether occluder body proximal end 103 to occluder body distal end 102 when occluder body 101 is in the compressed deployed configuration. Since that occluder body 101 is compressed elastically from the more elastically relaxed preliminary deployed configuration to the more elastically stressed compressed deployed configuration, tension member 106 is held tightened (taut) between a proximal connection portion of occluder body proximal end 103 and distal connection portion of occluder body distal end 102. Tension member 106 optionally includes or is configured as a rigid or flexible elongated member, optionally in a form of a rod or a wire, and it optionally includes at least one recess or tooth projecting angled to long axis of tension member 106. Tension member 106 is optionally fixated with a distal end thereof to occluder body distal end 102 (as shown), although it may be fixated to other portions of occluder body 101. Occluder body 101 is optionally configured to cover entire length of the tension member 106, when in the compressed deployed configuration.

In some embodiments, vascular occluder 100 is configured for placement inside a passage (e.g., a blood vessel such as an artery), particularly if the passage is equal to or smaller in diameter than occluder body 101 when in the preliminary deployed configuration and/or in the compressed deployed configuration. In some such embodiments, when in the compressed deployed configuration, occluder body 101 is configured to apply pressure to the wall portion (e.g., an arterial inner wall portion) thereby generating radial stress and/or friction sufficient for fixating vascular occluder 100 to the wall portion. When occluder body 101 is in the compressed deployed configuration, proximal tubular body segment 104 may form a structural insert that is configured to support (from within) distal tubular body segment 105, such that the resistance to radial inward compression of occluder body 101 is increased by about 10% or more, optionally about 20% or more, optionally 50% or more, or optionally by 100% or more, than when occluder body 101 is in the preliminary deployed configuration.

Vascular occluder 100 comprising a proximal locking portion 111 (shown in FIGS. 13 and 14) connected to and/or extending distally from occluder body proximal end 102. Proximal locking portion 111 is configured to engage and lock to a portion of tension member 106, when occluder body 101 is in the compressed deployed configuration. When locked, proximal locking portion 111 is configured to resist axial extension of occluder body 101 from the compressed deployed configuration. Proximal locking portion 111 includes an inner surface configured to engage one or more motion resisting discrete protuberances 112 (e.g., configured as a snap lock member or a ratchet, for example) emerging from a surface of tension member 106 so as to prevent axial motion (in a proximal direction) of proximal locking portion 111 relative to tension member 106.

Vascular occluder 100 also includes a pulling member 107 releasably connected or connectable to tension member 106 and configured to transfer a tension force from a proximal end thereof to tension member 106. Pulling member is optionally flexible and optionally formed of a Ni—Ti alloy wire. Pulling member 107 includes a connection element 107C at a distal end thereof configured to selectively connect to or disconnect from a mating connection element 106C provided at a proximal end of tension member 106. In some embodiments, connection element 107C and mating connection element 106C are connected by way of threading, screwing, or bolting (for example, connection element 107C includes a female threaded portion such as a nut and mating connection element 106C includes a male threaded portion such as a screw, or vice versa). Vascular occluder 100 may be provided (e.g., packaged) with connection element 107C and mating connection element 106C connected to each other, so the operator can disconnect them following occluder body 101 compression and deployment, such as by way of unthreading or unscrewing, via proximal end of pulling member 107, from outside the subject's body.

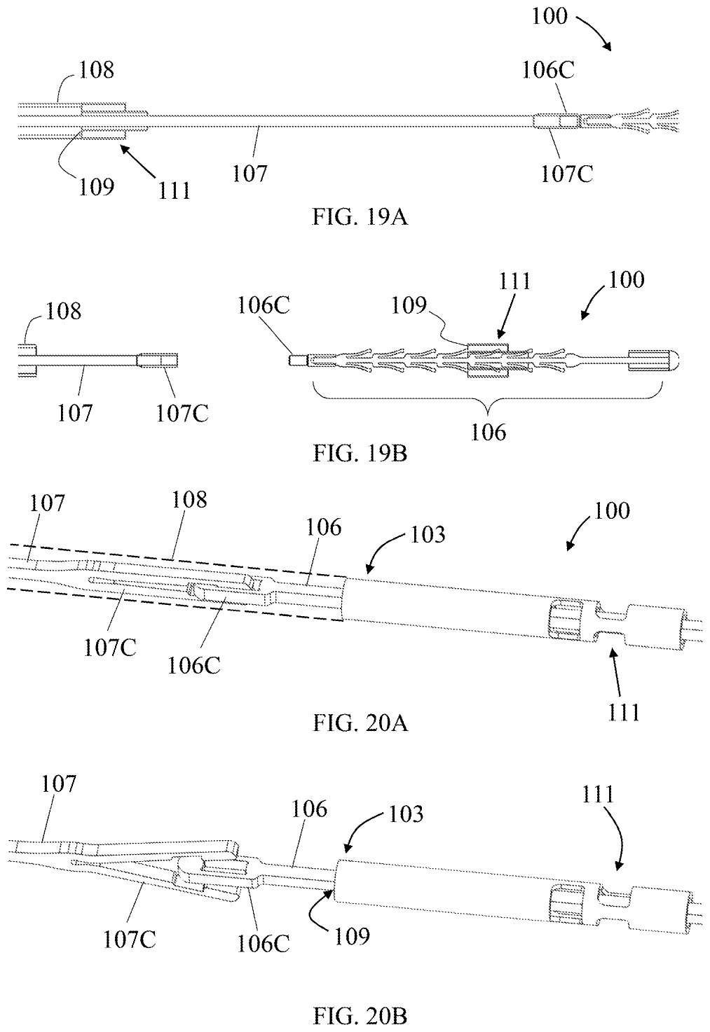

Vascular occluder 100, or a system or a kit comprising vascular occluder 100, may include an elongated pusher 108 that is configured to transfer a pushing force via a proximal end thereof (optionally from outside the subject's body) to occluder body proximal end 103. Pusher 108 encloses a lumen extending along its length, such as through a proximal opening located at its proximal end and through a distal opening located at its distal end. The pusher lumen is sized for allowing unhindered travel (e.g., by way of pulling) of pulling member 107 therethrough. Occluder body 101 includes a force receiving surface 109 (shown in FIGS. 19, for example) located at an inner portion of occluder body proximal end 103 and pointing proximally therefrom, configured to receive a pushing force from pusher 108 and for transferring it adequately for longitudinally compressing occluder body 101. Force receiving surface 109 also encloses an opening that is sufficiently sized for allowing unhindered travel of pulling member 107 and/or tension member 106 therethrough. Pusher 108 can be used for compressing occluder body 101 longitudinally from the preliminary deployed configuration to the compressed deployed configuration, such as by pushing against force receiving surface 109 and/or pulling tension member 106 with pulling member 106.

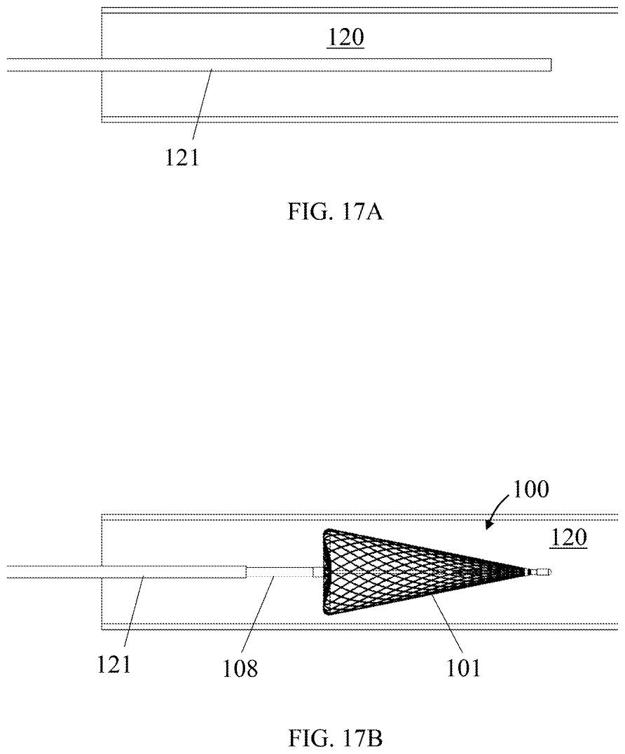

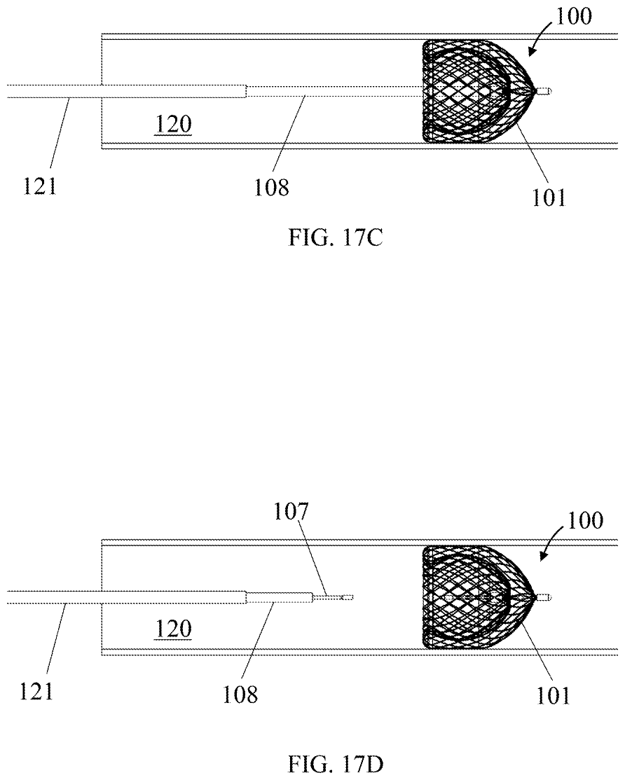

FIGS. 17A-17D illustrate side views of some exemplary scenarios in an exemplary method for deploying vascular occluder 100 in a passage 120 optionally formed by a blood vessel (e.g., artery) in a live subject. Blood vessel treatments may include applying vascular occluder 100 for treating or preventing hemorrhage, for blocking or stopping distal perfusion during embolization treatments and/or for treating or improving venous insufficiency of deep veins, for example. Passage 120 may have an inner diameter equal to or smaller than a maximal compressed deployed diameter of occluder body 101 when in the compressed deployed configuration, and the operator (user) may choose in advance a specific size of vascular occluder 100 accordingly to achieve certain dimensional relations. Preliminary steps before insertion of vascular occluder 100 may include accessing patient's vasculature using a vascular access kit optionally comprising an access needle, and/or an introducer sheath that can be advanced into the vasculature over the access needle, then introducing a guidewire through the introducer sheath (after removing the access needle therefrom) until reaching or passing the target location in passage 120. As shown in FIG. 17A, a delivery system such as a catheter 121 is first introduced into passage 120. Catheter 121 may be inserted with lumen thereof over an insertion assisting device such as a guidewire, which can be withdrawn before inserting pusher 108 with vascular occluder 101, or it can be inserted already with pusher 108 with vascular occluder 101 provided therein, or with its lumen free from any artifact. Once catheter 121 is properly positioned and patent at a chosen target location in passage 120, occluder body 101 can be pushed through catheter 121 into passage 120 by pushing pusher 108 against force receiving surface 109 of occluder body 101. While in catheter 121, occluder body 101 is maintained in a delivery configuration (as shown in FIG. 2A and FIG. 15A, for example) such that it is elastically stretched longitudinally.

Vascular occluder 100 can be released in passage 120 after occluder body 101 is fully extended and uncovered from catheter 121. Releasing of vascular occluder 100 may be performed by holding pusher 108 in-place and withdrawing (proximally) catheter 120, as shown in the figures, or by holding catheter 120 in-place and pushing (distally) vascular occluder 100 therefrom using pusher 108, or any combination between the options. Following or as part of its release, occluder body 101 can be allowed or caused to change into the preliminary deployed configuration. In the preliminary deployed configuration occluder body proximal end 103 is not connected or tethered to occluder body distal end 102, and tension member 106 is unconnected to occluder body proximal end 103 (as shown in FIG. 17B). In some embodiments, occluder body 101 in the preliminary deployed configuration is sized to allow repositioning in passage 120. In some such embodiments, the widest portion (e.g., its most proximal portion or base, optionally adjacent to crease 117) of occluder body 101 at its proximal end 103 may be sized such that it presses against wall portions enclosing passage 120, however in the preliminary deployed configuration occluder body 101 is sufficiently conformable and flexible and has small enough resistance to radial compression, so that with minimal or normal manual force it can be shifted along passage 120 without causing harm to surrounding passage wall portion.

As shown in FIG. 17C, after expanding to the preliminary deployed configuration, occluder body 101 can be compressed longitudinally using pulling member 107 and pusher 108, while having proximal tubular body segment 104 inverted, until occluder body 101 reaches and/or is locked in the compressed deployed configuration, such that tension member 106 tethers occluder body proximal end 103 to occluder body distal end 102 and held tightened therebetween, and occluder body 101 forms a radially expanded dome-like shape. If vascular occluder 100 is in an appropriate size relative to passage 120, then compressing occluder body 101 into the compressed deployed configuration is configured to directly cause a local increase in radial stress in a wall portion forming passage 120 and/or a local increase in diameter of passage 120, thereby anchoring thereto by way of continuous pressure and/or friction.

In some embodiments, passage 120 is a blood vessel (e.g., artery) in a live subject and vascular occluder 100 is configured to immediately or gradually occlude the blood vessel once occluder body 101 is locked in the compressed deployed configuration and anchors to passage 120 wall portion. Releasing occluder body 101 and/or allowing it to regain (a more elastically relaxed) preliminary deployed configuration, may cause at least partial reduction in blood flow in the artery, so changing it to the compressed deployed configuration can reduce overall permeability of occluder body 101 for blood flowing therethrough. Passage 120 occlusion may be immediate or gradual (e.g., several seconds, minutes, or more, until complete blockage). The operator may then decide if to remove vascular occluder 100 or keep it implanted. For implanting vascular occluder 100 in passage 120 when occluder body is in the compressed deployed configuration, pulling member 107 can be disconnected from tension member 106, and then be withdrawn with pusher 108 (FIG. 17D).

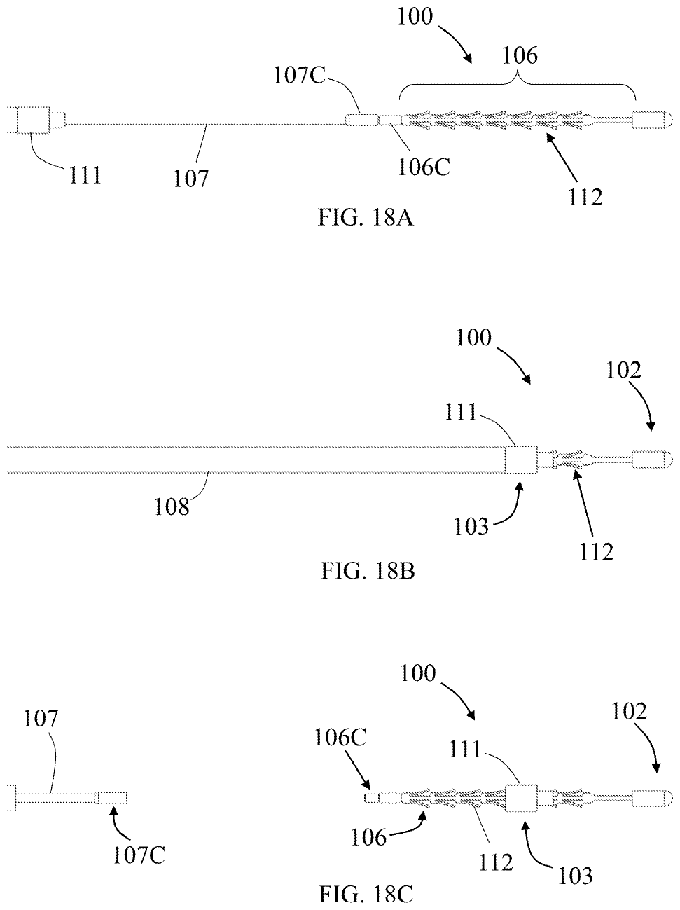

FIGS. 18A-18C illustrate side views, and FIGS. 19A-19B illustrate cross-sectional side views, of an exemplary compression mechanism of vascular occluder 100 in accordance with steps of the exemplary method shown in FIG. 17. FIG. 18A and FIG. 19A show an instance equivalent to the instance shown in FIG. 17B, when occluder body 101 is fully extended distally from catheter 121 and after changing to the preliminary deployed configuration. As shown, pulling member 107 is extending distally from pusher 108 and is connected to connection element 107C thereof to mating connection element 106C of tension member 106. As shown in this example, tension member 106 is a rigid machined member that comprises a plurality of opposing pairs of protuberances 112, each protuberance 112 is configured as a snap member oriented laterally and distally for allowing continuous advancement of proximal locking portion 111 across it in a proximal-to-distal direction, and to resist or prevent retraction of proximal locking portion 111 across it in a distal-to-proximal direction. FIG. 18B shows an instance equivalent to the instance shown in FIG. 17C, after compressing occluder body 101 to the compressed deployed configuration. As shown, pusher 108 was applied to push and advance distally occluder body proximal end 103 with proximal locking portion 111, while pulling member 107 under a force sufficient to keep occluder body distal end 102 in-place, until proximal locking portion snaped-locked to a chosen pair of protuberances 112 for locking occluder body 101 in the compressed deployed configuration. FIG. 18C and FIG. 19B show an instance equivalent to the instance shown in FIG. 17D, after pulling member 107 was disconnected (e.g., unscrewed) from tension member 106 and partly withdrawn with pusher 108 from the implanted vascular occluder 100.

FIGS. 20A-20B illustrate an exemplary release mechanism of vascular occluder 100 before and after activation thereof, respectively, which can be alternative to the mechanism shown in FIGS. 18 and 19. As shown in FIG. 20A, pusher 108 (schematically illustrated with dashed lines) is shown enclosing a distal portion of pulling member 107 and a proximal portion of tension member 106. Connection element 107C of pulling member 107 in this example is configured as a normally opened grasper-type element comprising two opposing grasper arms, and is configured to close on to mating connection element 106C of tension member 106 for tightly grasping thereto as needed. Pusher 108 and connection element 107C are sized and configured such that forced into a closed state if it is covered, partially or fully, with pusher 108 (as shown in FIG. 20A), and to elastically shift to an opened state (as shown in FIG. 20B) when pusher 108 is withdrawn therefrom thereby causing release of mating connection element 106C. in this example, pusher 108 can be applied for achieving two sequential outcomes: first to push occluder body proximal end 103 (at force receiving surface 109) until proximal locking portion 111 engages and fastens to tension member 106 when occluder body 101 is in the compressed deployed configuration, and then to be withdrawn sufficiently to cause disconnection of pulling member 107 from tension member 106.

Each of the following terms written in singular grammatical form: ‘a’, ‘an’, and ‘the’, as used herein, means ‘at least one’, or ‘one or more’. Use of the phrase ‘one or more’ herein does not alter this intended meaning of ‘a’, ‘an’, or ‘the’. Accordingly, the terms ‘a’, ‘an’, and ‘the’, as used herein, may also refer to, and encompass, a plurality of the stated entity or object, unless otherwise specifically defined or stated herein, or, unless the context clearly dictates otherwise. For example, the phrases: ‘a unit’, ‘a device’, ‘an assembly’, ‘a mechanism’, ‘a component’, ‘an element’, and ‘a step or procedure’, as used herein, may also refer to, and encompass, a plurality of units, a plurality of devices, a plurality of assemblies, a plurality of mechanisms, a plurality of components, a plurality of elements, and, a plurality of steps or procedures, respectively.

Each of the following terms: ‘includes’, ‘including’, ‘has’, ‘having’, ‘comprises’, and ‘comprising’, and, their linguistic/grammatical variants, derivatives, or/and conjugates, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably. For example, for stating or describing the numerical range of room temperature, the phrase ‘room temperature refers to a temperature in a range of between about 20° C. and about 25° C.’, and is considered equivalent to, and meaning the same as, the phrase ‘room temperature refers to a temperature in a range of from about 20° C. to about 25° C.’.

The term ‘about’, as used herein, refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment, may also be illustratively described and presented in the context or format of a plurality of separate embodiments.

Although the invention has been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, fall within the spirit of, and are encompassed by, the broad scope of the appended claims. All publications, patents, and or/and patent applications, cited or referred to in this disclosure are herein incorporated in their entirety by reference into the specification, to the same extent as if each individual publication, patent, or/and patent application, was specifically and individually indicated to be incorporated herein by reference. In addition, citation or identification of any reference in this specification shall not be construed or understood as an admission that such reference represents or corresponds to prior art of the present invention. To the extent that section headings are used, they should not be construed as necessarily limiting.

1. A vascular occluder, comprising:
an occluder body extending longitudinally between an occluder body distal end and an occluder body proximal end, the occluder body comprising a proximal tubular body segment and a distal tubular body segment, and wherein the occluder body is elastically compressible longitudinally from a preliminary deployed configuration to a compressed deployed configuration; and
a tension member configured to tether the occluder body proximal end to the occluder body distal end when the occluder body is in the compressed deployed configuration;
wherein, when the occluder body is in the compressed deployed configuration, the proximal tubular body segment is located inside of, and/or provides structural support to, the distal tubular body segment.

2. The vascular occluder according to claim 1, comprising a proximal occluding section extending between the occluder body proximal end and the proximal tubular body segment, and/or a distal occluding section extending between the occluder body distal end and the distal tubular body segment, configured to cover and/or at least partially occlude most or all cross-sectional area of a passage, when the occluder body is in the compressed deployed configuration.

3. The vascular occluder according to claim 2, wherein the proximal occluding section and/or the distal occluding section includes a meshed, braided and/or porous pattern and/or material across most or all area thereof, configured to reduce flow velocity, and/or to prevent localized increase of flow velocity, of blood passing therethrough, when the occluder body is in the compressed deployed configuration.

4. (canceled)

5. The vascular occluder according to claim 1, wherein, when the occluder body changes from the preliminary deployed configuration to the compressed deployed configuration, the proximal tubular body segment gradually and continuously becomes inserted into, and/or inwardly folded into, and/or tucked in, and/or stuffs, the distal tubular body segment, thereby forcing the distal tubular body segment to expand radially and/or to reshape elastically.

6. (canceled)

7. (canceled)

8. (canceled)

9. The vascular occluder according to claim 1, wherein the occluder body includes a portion between the occluder body proximal end and the distal tubular body segment comprising a crease and/or configured to facilitate a predetermined fold location and/or folding pattern when the proximal tubular body segment is pushed to advance towards the distal tubular body segment.

10. The vascular occluder according to claim 1, wherein the proximal tubular body segment is greater in average diameter than the distal tubular body segment, when the occluder body is in the preliminary deployed configuration.

11. The vascular occluder according to claim 1, wherein the proximal tubular body segment is greater in diameter than the distal tubular body segment along most or length thereof, when the occluder body is in the preliminary deployed configuration.

12. The vascular occluder according to claim 1, wherein a proximal portion of the proximal tubular body segment when the occluder body is in the preliminary deployed configuration, is configured to engage a distal portion of the distal tubular body segment when the occluder body is in the compressed deployed configuration.

13. The vascular occluder according to claim 1, wherein the occluder body reduces gradually in diameter in a direction from the occluder body proximal end to the occluder body distal end, when in the preliminary deployed configuration.

14. The vascular occluder according to claim 1, wherein at least part of the occluder body is cone-like shaped when in the preliminary deployed configuration and is dome-like shaped when in the compressed deployed configuration.

15. The vascular occluder according to claim 1, wherein the occluder body covers an entire length of the tension member when in the compressed deployed configuration.

16. The vascular occluder according to claim 1, wherein the tension member is fixated with a tension member distal end thereof to the occluder body distal end.

17. The vascular occluder according to claim 1, wherein the occluder body, when in the compressed deployed configuration, is configured to apply pressure to a wall portion enclosing a passage, thereby generating radial stress and/or friction sufficient for fixating the vascular occluder to the wall portion, wherein the passage is equal to or smaller in diameter than the occluder body when in the preliminary deployed configuration.

18. The vascular occluder according to claim 1, wherein a resistance to radial inward compression of the occluder body when changing from the preliminary deployed configuration to the compressed deployed configuration is increased by at least 10%.

19. (canceled)

20. (canceled)

21. (canceled)

22. The vascular occluder according to claim 19, further comprising a puling member, wherein the pulling member includes a connection element at a distal end thereof configured to selectively connect to or disconnect from a mating connection element provided at a proximal end of the tension member.

23. (canceled)

24. (canceled)

25. (canceled)

26. The vascular occluder according to claim 1, comprising a proximal locking portion connected to and/or extending from the occluder body proximal end, configured to engage a portion of the occluder body or the tension member, when the occluder body is in the compressed deployed configuration, and to resist axial extension of the occluder body from the compressed deployed configuration.

27. The vascular occluder according to claim 26, wherein the proximal locking portion includes an inner surface configured to engage one or more motion resisting discrete recesses or protuberances emerging from a surface of tension member, and/or to lock to the tension member by way of friction with an outer surface of the tension member, so as to resist and/or prevent axial motion of the proximal locking portion relative to the tension member.

28. The vascular occluder according to claim 1, wherein the occluder body is elastically stretchable longitudinally into a delivery configuration having a delivery length and a delivery diameter, wherein the occluder body, when in the preliminary deployed configuration, has a preliminary deployed length smaller than the delivery length and a preliminary deployed diameter greater than the delivery diameter.

29. (canceled)

30. (canceled)

31. (canceled)

32. The vascular occluder according to claim 1, wherein the occluder body is at least partially meshed, woven and/or braided.

33. The vascular occluder according to claim 1, wherein the occluder body is at least partially impermeable and/or is covered or impregnated with an impermeable or hydrophilic substance or material.

34-44. (canceled)
